(19) United States
(12) Patent Application Publication
(10) Pub. No.: US 2025/0057549 A1
(43) Pub. Date: Feb. 20, 2025

(54) APPARATUS AND METHODS FOR TREATING A TOTAL OCCLUSION IN A BLOOD VESSEL

(71) Applicant: Boston Scientific Scimed, Inc., Maple Grove, MN (US)

(72) Inventors: Gordon John Kocur, Lino Lakes, MN (US); Jeffrey Steven Fuller, Brooklyn Park, MN (US); Jeffrey Adam Hammerstrom, New Hope, MN (US); Kyle Logan Lemke, Hudson, WI (US)

(73) Assignee: Boston Scientific Scimed, Inc., Maple Grove, MN (US)

(21) Appl. No.: 18/801,979

(22) Filed: Aug. 13, 2024

Related U.S. Application Data
(60) Provisional application No. 63/532,475, filed on Aug. 14, 2023.

(51) Int. Cl. A61B 17/22; A61B 17/00

(52) U.S. Cl. CPC A61B 17/22; A61B 17/00234; A61B 2017/00238; A61B 2017/22051; A61B 2017/22077; A61B 2017/22079; A61B 2017/22095; A61B 2217/005

(57) ABSTRACT

An apparatus for treating a total occlusion in a blood vessel may include a self-orienting device including an elongate shaft and an inflatable orienting element. The shaft includes an inflation lumen having a first noncircular shape and a working lumen having a second noncircular shape. The inflatable orienting element includes first and second inflatable members extending laterally from the shaft. An outer diameter of the shaft is 0.045 inches or less, a cross-sectional area of the first noncircular cross-sectional shape is at least 0.00013 square inches, and a cross-sectional area of the second noncircular cross-sectional shape is at least 0.00056 square inches. The shaft may include a distal tail extending distally from the inflatable orienting element, the distal tail being at least 15 millimeters long. The shaft may include a plurality of first apertures communicating with the working lumen and disposed in the distal tail.

### CROSS-REFERENCE TO RELATED APPLICATIONS

This application claims the benefit of U.S. Provisional Patent Application Ser. No. 63/532,475, filed Aug. 14, 2023, which is incorporated herein by reference.

### TECHNICAL FIELD

The disclosure is directed to devices and methods for recanalization of an occluded blood vessel. More particularly, the disclosure is directed to devices and methods for re-entry into the true lumen from the extraluminal or subintimal space of a blood vessel.

### BACKGROUND

Chronic total occlusion (CTO) is an arterial vessel blockage that obstructs blood flow through the vessel, and can occur in both coronary and peripheral arteries. In some instances, it may be difficult or impossible to pass through the CTO with a medical device in an antegrade direction to recanalize the vessel. Accordingly, techniques have been developed for creating a subintimal pathway (i.e., a pathway between the intimal and adventitial tissue layers of the vessel) around the occlusion and then re-entering the true lumen of the vessel distal of the occlusion in an attempt to recanalize the vessel. In some instances, re-entering the true lumen from the subintimal space and/or recanalization can be difficult. For example, visualization of the true lumen may be difficult, which may make directing the re-entry device toward the true lumen challenging. Additionally, subintimal hematoma may further complicate re-entry into the true lumen, and in some instances may result in failure of the re-entry procedure. Accordingly, it is desirable to provide alternative recanalization devices for subintimal advancement and re-entry and/or associated methods of recanalizing a blood vessel in which a CTO is present. It is also desirable to minimize and/or treat hematoma during the procedure to reduce re-entry failure and/or other complications.

### SUMMARY

This disclosure provides design, material, manufacturing method, and use alternatives for medical devices facilitating re-entry into the true lumen from the extraluminal or subintimal space of a blood vessel.

A first example is an apparatus for treating a total occlusion in a blood vessel. The apparatus includes a self-orienting device comprising an elongate shaft, an inflatable orienting element disposed on a distal portion of the elongate shaft, and a manifold disposed at a proximal end of the elongate shaft. The elongate shaft includes a working lumen extending from the manifold to a distal facing port at a distal end of the elongate shaft. The elongate shaft includes an inflation lumen in fluid communication with the inflatable orienting element and the manifold. The inflatable orienting element includes a first inflatable member extending laterally from a central longitudinal axis of the elongate shaft in a first direction and a second inflatable member extending laterally from the central longitudinal axis of the elongate shaft in a second direction different from the first direction. In a proximal portion of the elongate shaft proximal of the inflatable orienting element, the inflation lumen has a first noncircular cross-sectional shape and the working lumen has a second noncircular cross-sectional shape. An outer diameter of the proximal portion of the elongate shaft is 0.045 inches or less. A cross-sectional area of the first noncircular cross-sectional shape is at least 0.00013 square inches. A cross-sectional area of the second noncircular cross-sectional shape is at least 0.00056 square inches.

Alternatively or additionally to any of the examples above, in another example, the elongate shaft comprises a first aperture communicating with the working lumen and opening in a third direction different from the first direction and the second direction. The first aperture is disposed between a proximal end of the inflatable orienting element and a distal end of the inflatable orienting element.

Alternatively or additionally to any of the examples above, in another example, the elongate shaft comprises a second aperture communicating with the working lumen and opening in a fourth direction different from the third direction. The second aperture is disposed between the proximal end of the inflatable orienting element and the distal end of the inflatable orienting element at a location longitudinally spaced apart from the first aperture.

Alternatively or additionally to any of the examples above, in another example, the proximal portion of the elongate shaft includes a proximal section wherein the inflation lumen has the first noncircular cross-sectional shape and the working lumen has the second noncircular cross-sectional shape, and a distal section wherein the inflation lumen and the working lumen each have circular cross-sectional shapes. A length of the proximal section is at least 50% of an overall length of the proximal portion of the elongate shaft.

Alternatively or additionally to any of the examples above, in another example, the first noncircular cross-sectional shape is crescent shaped.

Alternatively or additionally to any of the examples above, in another example, the second noncircular cross-sectional shape has a first maximum extent extending parallel to a major dimension of the first noncircular cross-sectional shape and a second maximum extent oriented perpendicular to the first maximum extent. The second maximum extent is less than the first maximum extent.

Alternatively or additionally to any of the examples above, in another example, the first maximum extent is greater than the major dimension.

Alternatively or additionally to any of the examples above, in another example, the inflatable orienting element is a first inflatable orienting element and the self-orienting device further comprises a second inflatable orienting element axially spaced apart from the first inflatable orienting element. The second inflatable orienting element includes a first inflatable member extending laterally from a central longitudinal axis of the elongate shaft in the first direction and a second inflatable member extending laterally from the central longitudinal axis of the elongate shaft in the second direction.

Alternatively or additionally to any of the examples above, in another example, the apparatus further includes an intermediate segment disposed between the first inflatable orienting element and the second inflatable orienting element. The intermediate segment includes a plurality of apertures opening in a plurality of directions different from the first direction and the second direction.

Alternatively or additionally to any of the examples above, in another example, the apparatus further includes a re-entry device configured to be slidably advanced within the working lumen. The elongate shaft is configured to direct the re-entry device toward a true lumen of the blood vessel at a position distal of the total occlusion.

Another example is an apparatus for treating a total occlusion in a blood vessel. The apparatus includes a self-orienting device comprising an elongate shaft, an inflatable orienting element disposed on a distal portion of the elongate shaft, and a manifold disposed at a proximal end of the elongate shaft. The elongate shaft includes a working lumen extending from the manifold to a distal facing port at a distal end of the elongate shaft. The elongate shaft includes an inflation lumen in fluid communication with the inflatable orienting element and the manifold. The inflatable orienting element includes a first inflatable member extending laterally from a central longitudinal axis of the elongate shaft in a first direction and a second inflatable member extending laterally from the central longitudinal axis of the elongate shaft in a second direction opposite the first direction. The elongate shaft comprises a distal tail extending distally from the inflatable orienting element to the distal end of the elongate shaft. The distal tail is at least 15 millimeters long. The elongate shaft includes a plurality of first apertures communicating with the working lumen and opening in a third direction different from the first direction and the second direction. The plurality of first apertures is disposed in the distal tail distal of the inflatable orienting element and proximal of the distal end.

Alternatively or additionally to any of the examples above, in another example, the elongate shaft includes a plurality of second apertures communicating with the working lumen and opening in a fourth direction opposite the third direction. The plurality of second apertures is disposed in the distal tail distal of the inflatable orienting element and proximal of the distal end.

Alternatively or additionally to any of the examples above, in another example, the plurality of second apertures is longitudinally spaced apart from the plurality of first apertures.

Alternatively or additionally to any of the examples above, in another example, in a proximal portion of the elongate shaft proximal of the inflatable orienting element, the inflation lumen has a first noncircular cross-sectional shape and the working lumen has a second noncircular cross-sectional shape. An outer diameter of the proximal portion of the elongate shaft is 0.045 inches or less. A cross-sectional area of the first noncircular cross-sectional shape is at least 0.00013 square inches. A cross-sectional area of the second noncircular cross-sectional shape is at least 0.00056 square inches.

Another example is a method of treating a total occlusion in a blood vessel. The method includes advancing a self-orienting device into a subintimal space within a wall of the blood vessel adjacent the total occlusion. The self-orienting device includes an elongate shaft, an inflatable orienting element disposed on a distal portion of the elongate shaft, and a manifold disposed at a proximal end of the elongate shaft. The elongate shaft includes a working lumen extending from the manifold to a distal facing port at a distal end of the elongate shaft, and an inflation lumen in fluid communication with the inflatable orienting element and the manifold. The inflatable orienting element includes a first inflatable member extending laterally from a central longitudinal axis of the elongate shaft in a first direction and a second inflatable member extending laterally from the central longitudinal axis of the elongate shaft in a second direction different from the first direction. The elongate shaft includes a distal tail extending distally from the inflatable orienting element to the distal end of the elongate shaft. The distal tail is at least 15 millimeters long. The elongate shaft includes a plurality of first apertures communicating with the working lumen and opening in a third direction different from the first direction and the second direction. The plurality of first apertures is disposed in the distal tail distal of the inflatable orienting element and proximal of the distal end. The inflatable orienting element is positioned radially outward of the total occlusion within the subintimal space such that the inflatable orienting element longitudinally overlaps the total occlusion and the distal tail extends distal of the total occlusion within the subintimal space. Blood is aspirated from within the subintimal space through the working lumen. The inflatable orienting element is inflated within the subintimal space such that the first inflatable member and the second inflatable member cooperate with the wall of the blood vessel to orient the plurality of first apertures toward a true lumen of the blood vessel.

Alternatively or additionally to any of the examples above, in another example, the method includes advancing a re-entry device within the working lumen and out one aperture of the plurality of first apertures into the true lumen of the blood vessel.

Alternatively or additionally to any of the examples above, in another example, the re-entry device is disposed within the working lumen prior to advancing the self-orienting device into a subintimal space within a wall of the blood vessel adjacent the total occlusion.

Alternatively or additionally to any of the examples above, in another example, the method includes aspirating blood from within the subintimal space occurs while the re-entry device is disposed within the working lumen.

Alternatively or additionally to any of the examples above, in another example, aspirating blood from within the subintimal space includes aspirating blood through the distal facing port and the plurality of first apertures.

Alternatively or additionally to any of the examples above, in another example, aspirating blood from within the subintimal space occurs while positioning the inflatable orienting element radially outward of the total occlusion within the subintimal space.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIGS. 1-3 are partial cross-sectional views of a blood vessel having a total occlusion disposed therein, and illustrating selected aspects of a crossing device treating the blood vessel via subintimal space;

FIG. 4 is a partial cross-sectional view of the blood vessel of FIGS. 1-3 with a hematoma formed distal of the total occlusion as a result of the crossing device accessing the subintimal space;

FIG. 5 is a perspective view illustrating selected aspects of a self-orienting device;

FIG. 6 illustrates selected aspects of the self-orienting device of FIG. 5 positioned within the subintimal space;

FIG. 7 illustrates selected aspects of a re-entry device disposed within the self-orienting device failing to re-enter the true lumen of the blood vessel due to the hematoma;

FIG. 8 illustrates selected aspects of a source of suction coupled to the self-orienting device;

FIG. 9 illustrates selected aspects of removing the hematoma via suction applied to the self-orienting device;

FIG. 10 illustrates selected aspects of a re-entry device disposed within the self-orienting device re-entering the true lumen of the blood vessel after removal of the hematoma;

FIG. 11A-11C illustrate examples of dual lumen shafts associated with the self-orienting device;

FIG. 12 is a perspective view illustrating selected aspects of the self-orienting device of FIG. 5;

FIG. 13 illustrates selected aspects of the self-orienting device of FIG. 5;

FIG. 14 illustrates selected aspects of the self-orienting device with cross-sectional cuts made at planes 14-14 and A-A in FIG. 13;

FIG. 15 illustrates selected aspects of the self-orienting device of FIG. 14;

FIGS. 16-17 illustrate alternative configurations of the self-orienting device of FIG. 5;

FIG. 18 illustrates an alternative configuration of the self-orienting device of FIG. 5;

FIG. 19 illustrates an alternative configuration of the self-orienting device of FIG. 18;

FIG. 20-21 illustrates using the self-orienting device of FIG. 18 to re-enter the true lumen of the blood vessel distal of the hematoma;

FIG. 22 illustrates using the self-orienting device of FIG. 18 to re-enter the true lumen of the blood vessel distal of the total occlusion;

FIG. 23A is a partial cross-sectional view illustrating selected aspects of a self-orienting device;

FIG. 23B is a partial cross-sectional view illustrating selected aspects of the self-orienting device of FIG. 23A positioned within a hematoma;

FIG. 24A is a partial cross-sectional view illustrating selected aspects of a self-orienting device;

FIG. 24B is a partial cross-sectional view illustrating selected aspects of the self-orienting device of FIG. 24A positioned within a hematoma;

FIG. 25 illustrates selected aspects of an alternative configuration of a self-orienting device;

FIG. 26A is a partial cross-sectional view illustrating selected aspects of a self-orienting device; and

FIGS. 26B-C are partial cross-sectional views illustrating selected aspects of the self-orienting device of FIG. 26A positioned within a hematoma.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

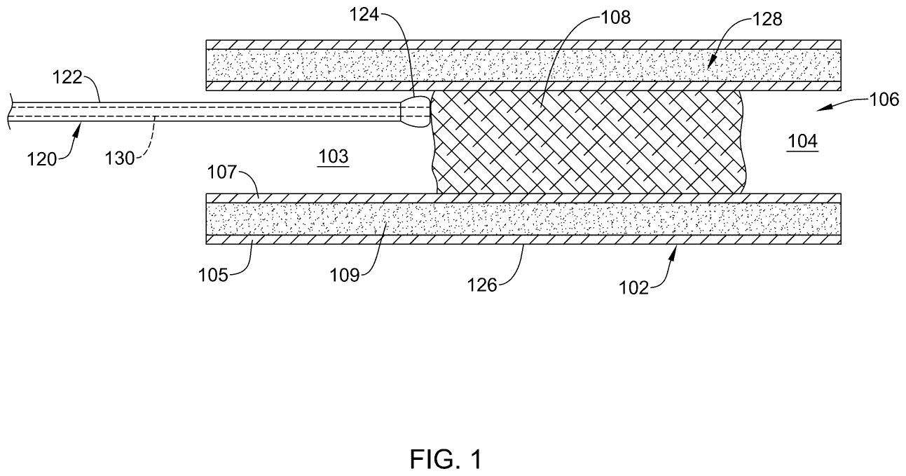

FIG. 1 is a partial cross-sectional view illustrating selected aspects of a blood vessel 102 (e.g., an artery) having a wall 126. The wall 126 of the blood vessel 102 may comprise three layers—an outermost layer 105 known as the adventitia, an innermost layer 107 known as the intima, and tissue(s) 109 extending between the outermost layer 105 and the innermost layer 107 collectively referred to as the media. For the purpose of illustration, the three layers of the wall 126 are each shown as a single homogenous layer in FIG. 1. However, in the human body, the innermost layer 107 (e.g., the intima) and the tissue(s) 109 (e.g., the media) may each comprise a plurality of sub-layers. The innermost layer 107 (e.g., the intima) is a layer of endothelial cells lining the true lumen 106 of the blood vessel 102, as well as a subendothelial layer made up of mostly loose connective tissue. The tissue(s) 109 (e.g., the media) is a muscular layer formed primarily of circumferentially arranged smooth muscle cells. The outermost layer 105 (e.g., the adventitia) of the wall 126 is formed primarily of loose connective tissue made up of fibroblasts and associated collagen fibers. In some embodiments, subintimal space 128 may be considered to extend from the innermost layer 107 (e.g., the intima) to the outermost layer 105 (e.g., the adventitia). In some embodiments, the transition between the outermost portion of the innermost layer 107 (e.g., the intima) and the innermost portion of the tissue(s) 109 (e.g., the media), may be referred to as the subintimal space.

The innermost layer 107 (e.g., the intima) may define a true lumen 106 of the blood vessel 102. In FIG. 1, a total occlusion 108 is shown disposed in and/or blocking the true lumen 106 of the blood vessel 102. The total occlusion 108 may divide the true lumen 106 of the blood vessel 102 into a proximal segment 103 and a distal segment 104.

In some instances, it may be undesired, difficult, or impossible to pass through the total occlusion 108 with a medical device to recanalize the blood vessel 102. In such instances, it may be possible to recanalize the blood vessel 102 through a subintimal approach. One such technique is called Antegrade Dissection and Reentry (ADR). A crossing device 120 is shown in FIG. 1 disposed within the proximal segment 103 of the true lumen 106 of the blood vessel 102. The crossing device 120 may comprise an elongate tubular member 122 (e.g., a shaft, a hypotube, etc.) having a lumen 130 extending therein.

In some embodiments, the crossing device 120 and/or the elongate tubular member 122 may comprise a coil, a braided structure, or other support element(s). In some embodiments, the crossing device 120 and/or the elongate tubular member 122 may comprise a plurality of filars that are wound in a generally helical shape. In some embodiments, the crossing device 120 and/or the elongate tubular member 122 may comprise a polymeric sleeve (e.g., heat shrink tubing, a polymer layer, etc.) disposed on and/or about at least a portion of the elongate tubular member 122 (e.g., the coil, the braided structure, etc.). In some embodiments, the crossing device 120 and/or the elongate tubular member 122 may comprise a helical cut or a plurality of helical cuts along and/or within a distal portion thereof. In some embodiments, the helical cut or the plurality of helical cuts may be dimensioned and/or shaped to provide an advantageous transition in lateral stiffness within the crossing device 120 and/or the elongate tubular member 122. In some embodiments, the crossing device 120 and/or the elongate tubular member 122 may comprise a proximal hub fixed at a proximal end of the crossing device 120 and/or the elongate tubular member 122. The proximal hub may comprise Luer fitting.

In some embodiments, the crossing device 120 may comprise a distal tip 124 fixed at and/or to a distal end of the elongate tubular member 122. In some embodiments, the distal tip 124 may have a generally rounded shape. In some embodiments, the distal tip 124 may be a bulbous distal tip. In some embodiments, the distal tip 124 may be incapable of cutting and/or piercing the innermost layer 107 (e.g., the intima) of the blood vessel 102 and/or the wall 126. In some embodiments, the distal tip 124 may include an outer surface that is relatively smooth and/or generally nonabrasive. In some embodiments, the distal tip 124 may be formed from a suitable metallic material. In some embodiments, the distal tip 124 may be formed form a suitable polymeric material. Some nonlimiting examples of suitable materials are disclosed below.

In some embodiments, the distal tip 124 may include one or more radiopaque markers. In some embodiments the one or more radiopaque markers may be disposed on the elongate tubular member 122 underneath and/or radially inward of the distal tip 124. In some embodiments the one or more radiopaque markers may be embedded within the distal tip 124. In some embodiments the one or more radiopaque markers may be disposed on the outer surface and/or exposed to an exterior of the distal tip 124. Other configurations are also contemplated.

In FIG. 1, the crossing device 120 is shown approaching and/or contacting the total occlusion 108. A practitioner may discover, via a probing motion, where the total occlusion 108 is located and/or that the total occlusion 108 cannot be crossed within the true lumen 106. The practitioner may then seek to implement a recanalization procedure via the subintimal space 128. In some embodiments, crossing the total occlusion 108 via the subintimal space 128 may include advancing a guidewire (not shown) within the lumen 130 of the crossing device 120, out the distal tip 124, and into or through the innermost layer 107 (e.g., the intima) of the blood vessel 102 and/or the wall 126 to the subintimal space 128. The lumen 130 may be sized and configured to permit slidable movement of the guidewire therein and/or relative to the crossing device 120 and/or the elongate tubular member 122.

In some embodiments, the guidewire may be advanced into the proximal segment 103 of the true lumen 106 of the blood vessel 102 and into the subintimal space 128 prior to inserting and/or advancing the crossing device 120 into the blood vessel 102. In such embodiments, the crossing device 120 may be advanced over the guidewire into the proximal segment 103 of the true lumen 106 of the blood vessel 102 and then into the subintimal space 128 while tracking over the guidewire.

In some embodiments, the guidewire may be advanced into the proximal segment 103 of the true lumen 106 of the blood vessel 102 and into the subintimal space 128 after inserting and/or advancing the crossing device 120 into the blood vessel 102. The guidewire may be advanced into the proximal segment 103 of the true lumen 106 within the lumen 130 of the crossing device 120. The guidewire may then the advanced out the distal tip 124 of the crossing device 120 and into the innermost layer 107 (e.g., the intima) of the blood vessel 102 and/or the wall 126. Upon gaining access to the subintimal space 128 with the guidewire, the crossing device 120 may be advanced over the guidewire into the subintimal space 128 and alongside the total occlusion 108.

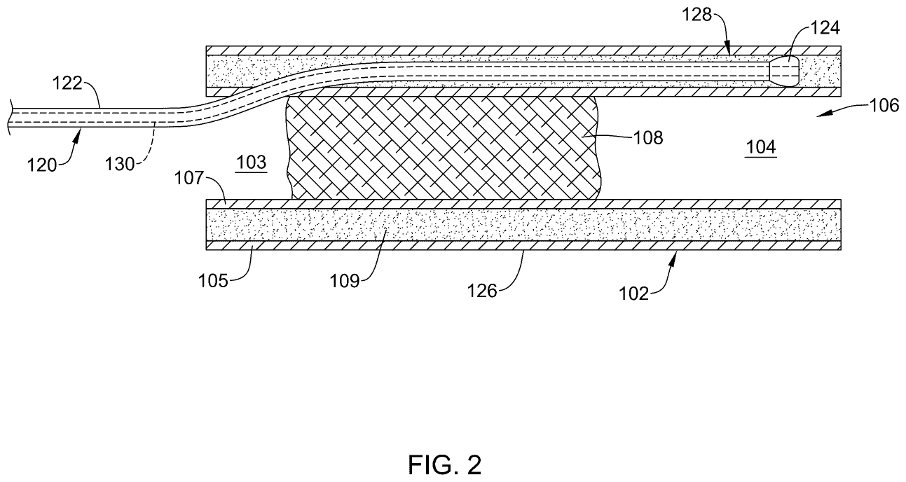

The crossing device 120 may be further advanced, over the guidewire for example, within the subintimal space 128 until the distal tip 124 has advanced past the total occlusion 108, as seen in FIG. 2. In some embodiments, a relative position of the crossing device 120 with respect to the total occlusion 108 may be determined by an appropriate imaging technique. In some embodiments, the lumen 130 of the crossing device 120 may be used to deliver fluids (e.g., a radiopaque fluid, a contrast agent, a therapeutic agent, etc.) into the body. In some embodiments, the lumen 130 of the crossing device 120 may be used to deliver fluids (e.g., a radiopaque fluid, a contrast agent, a therapeutic agent, etc.) into the subintimal space 128. Other configurations are also contemplated.

In some embodiments, the crossing device 120 may be rotated about its longitudinal axis as the crossing device 120 is advanced axially. In some embodiments, rotation of the crossing device 120 may reduce resistance to axial advancement of the crossing device 120 within the subintimal space 128. In some embodiments, after advancing the distal tip 124 of the crossing device 120 distal of the total occlusion 108, the guidewire may be held in place and the crossing device 120 may be removed from the subintimal space 128 and/or the blood vessel 102. The guidewire may be left in place to function as a guide and/or may be used to track an orienting device into the subintimal space 128.

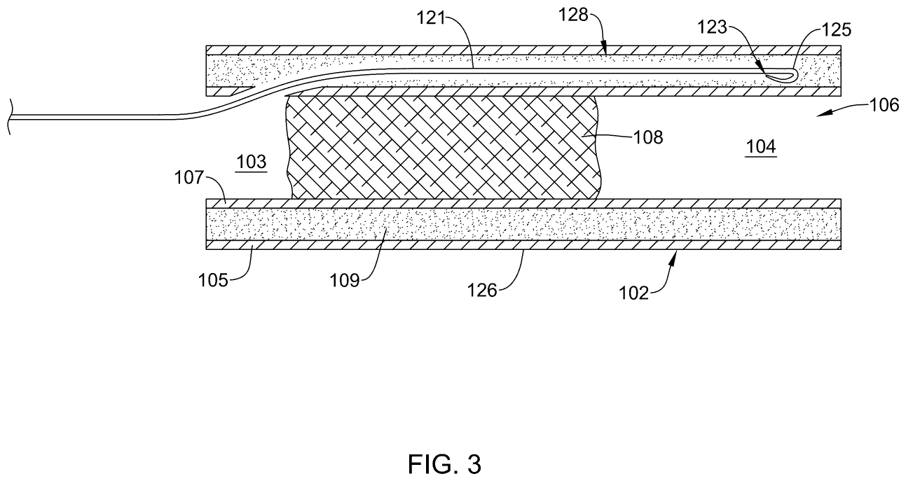

In some embodiments the crossing device may be a guidewire 121 having a distal end 123 designed to prolapse and create a distal loop or a knuckle 125 configured to create a dissection plane within the subintimal space 128 as the knuckle 125 is advanced distally within the subintimal space 128, as seen in FIG. 3. This is referred to as a knuckle wire technique.

Axial advancement of the distal tip 124 and/or the crossing device 120 and/or the guidewire 121 within the subintimal space 128 may cause blunt dissection of the layers of the wall 126 of the blood vessel 102. Axial advancement of the distal tip 124 and/or the crossing device 120 and/or the guidewire 121 within the subintimal space 128 may provide a path for recanalization of the blood vessel 102. In some situations, the knuckle wire technique may utilize a crossing device (e.g., the guidewire 121) that is cheaper to purchase but makes the dissection path/plane and/or distal advancement of the crossing device harder to control. In some instances, the knuckle wire technique may produce a dissection path/plane that spirals and/or rotates around the true lumen 106 as the guidewire 121 is advanced distally instead of advancing generally parallel to the true lumen 106.

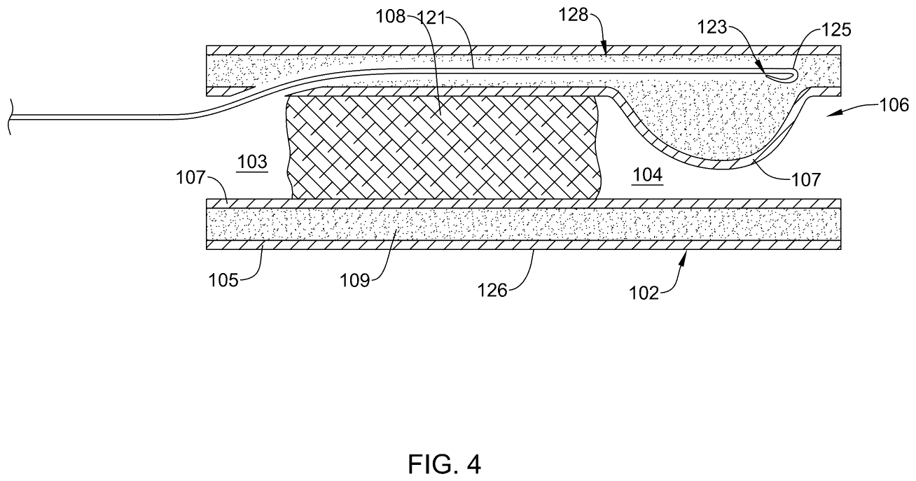

Additionally, in some cases, the total occlusion 108 may cause higher pressure to form within the proximal segment 103 of the true lumen 106 of the blood vessel 102 (e.g., upstream of the total occlusion 108 in an artery) and lower pressure to form within the distal segment 104 of the true lumen 106 of the blood vessel 102 (e.g., downstream of the total occlusion 108 in an artery). As a result, blood may be forced into the subintimal space 128 and/or between the layers of the wall 126 of the blood vessel 102 that were dissected by the crossing device 120. The lower pressure in the distal segment 104 of the true lumen 106 of the blood vessel 102 (e.g., the pressure differential) may cause the innermost layer 107 (e.g., the intima) of the wall 126 to collapse radially inward toward and/or into the distal segment 104 of the true lumen 106 of the blood vessel 102 and/or may form a hematoma 110 within the wall 126 of the blood vessel 102, as shown in FIG. 4, as the subintimal space 128 fills with blood from the proximal segment 103. The hematoma 110 may at least partially close off the distal segment 104 of the true lumen 106 of the blood vessel 102 distal of the total occlusion 108. The hematoma 110 may also make re-entry into the true lumen 106 of the blood vessel 102 more difficult, and in some cases may cause re-entry failure. Hematoma formation may follow dissection of the wall 126 of the blood vessel 102 regardless of which type of crossing device is used, but some data suggests that hematoma formation may be more prevalent when using the knuckle wire technique.

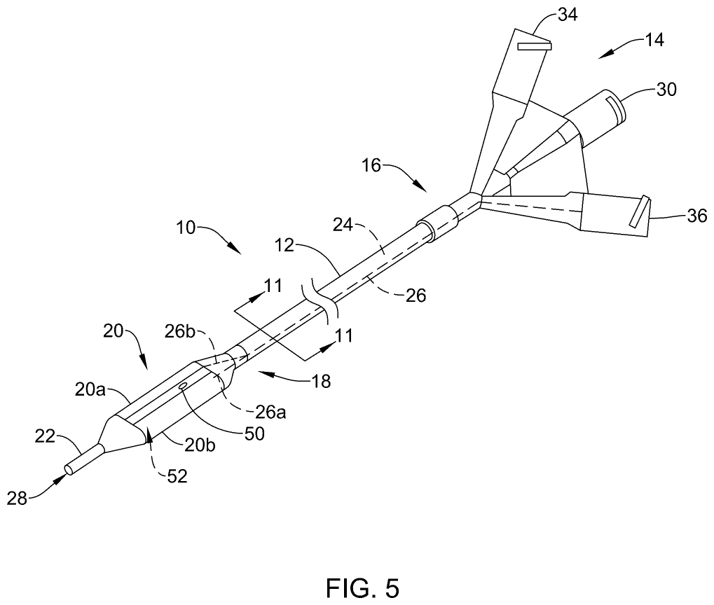

FIG. 5 is a schematic view illustrating selected aspects of a self-orienting device 10 of an apparatus for treating the total occlusion 108 in the blood vessel 102. The self-orienting device 10 may comprise an elongate shaft 12 extending from a manifold 14 at a proximal end 16 of the elongate shaft 12 to an inflatable orienting element 20 disposed on and/or mounted on a distal portion 18 of the elongate shaft 12. In some embodiments, the distal portion 18 of the elongate shaft 12 may extend through the inflatable orienting element 20 to form a distal tip 22 extending distal of the inflatable orienting element 20.

The self-orienting device 10 and/or the elongate shaft 12 may comprise a working lumen 24 extending from the manifold 14 to a distal facing port 28 at a distal end of the elongate shaft 12 and/or the distal tip 22. In some embodiments, the manifold 14 may comprise a first port 30 in communication with the working lumen 24. In some embodiments, the first port 30 may be oriented generally parallel to the working lumen 24. In some embodiments, the first port 30 may be coaxially aligned with the working lumen 24. Other configurations are also contemplated. The first port 30 and/or the working lumen 24 may be sized and configured to slidably receive a medical device (e.g., a guidewire, a re-entry device, etc.). In some embodiments, the first port 30 may be considered a device port. In some embodiments, the medical device (e.g., the guidewire, the re-entry device, etc.) may be rotatable within the first port 30 and/or the working lumen 24. In some alternative configurations, the medical device (e.g., the guidewire, the re-entry device, etc.) may be nonrotatable within the first port 30 and/or the working lumen 24.

In some embodiments, the manifold 14 may comprise a second port 34 in fluid communication with the working lumen 24. In some embodiments, the second port 34 may be oriented at an oblique angle to the working lumen 24. Other configurations are also contemplated. In some embodiments, the second port 34 may be useful for transferring fluid to and/or from the working lumen 24 with and/or without the medical device (e.g., the guidewire, the re-entry device, etc.) disposed within the first port 30 and/or the working lumen 24. In some embodiments, the second port 34 may be useful for aspiration of the hematoma 110, as described herein. In some embodiments, the second port 34 may be considered an aspiration port. The second port 34 may be configured to be fluidly connected to a source of suction.

In some embodiments, the self-orienting device 10 and/or the elongate shaft 12 may be configured to be advanced over a guidewire for delivery to a remote location in the vasculature of a patient (e.g., to and/or adjacent the total occlusion 108, for example). In some embodiments, the self-orienting device 10 and/or the elongate shaft 12 may be configured as an over-the-wire (OTW) catheter having the working lumen 24 extending through the entire length of the self-orienting device 10 and/or the elongate shaft 12 from the distal facing port 28 at the distal tip 22 to the first port 30 in the manifold 14. In some embodiments, the distal facing port 28 may be disposed at a distalmost extent of the self-orienting device 10 and/or the elongate shaft 12.

The self-orienting device 10 and/or the elongate shaft 12 may comprise an inflation lumen 26 in fluid communication with the inflatable orienting element 20 and the manifold 14. In some embodiments, the manifold 14 may comprise a third port 36 in fluid communication with the inflation lumen 26. The third port 36 may be configured to fluidly connect to a source of inflation fluid (not shown). In some embodiments, the third port 36 may be considered an inflation port.

In some embodiments, the inflatable orienting element 20 may comprise a first inflatable member 20a extending laterally from a central longitudinal axis of the elongate shaft 12 in a first direction, and a second inflatable member 20b extending laterally from the central longitudinal axis of the elongate shaft 12 in a second direction different from the first direction. In some embodiments, the second direction may be opposite the first direction. In some embodiments, the first inflatable member 20a and/or the second inflatable member 20b may be integrally formed and/or monolithically formed with the elongate shaft 12. In some embodiments, an outer surface of the elongate shaft 12 may be exposed to an exterior of the self-orienting device 10 between the first inflatable member 20a and the second inflatable member 20b. Other configurations are also contemplated.

In some embodiments, the self-orienting device 10 and/or the elongate shaft 12 may comprise a first aperture 50 communicating with the working lumen 24 and opening to the exterior of the self-orienting device 10 and/or the elongate shaft 12 in a third direction different from the first direction and the second direction. In some embodiments, the third direction may be substantially perpendicular to the first direction and the second direction. Other configurations are also contemplated. In some embodiments, the first aperture 50 may be disposed between a proximal end of the inflatable orienting element 20 and a distal end of the inflatable orienting element 20.

In some embodiments, the self-orienting device 10 and/or the elongate shaft 12 may comprise a second aperture 52 communicating with the working lumen 24 and opening to the exterior of the self-orienting device 10 and/or the elongate shaft 12 in a fourth direction different from the first direction and the second direction. In some embodiments, the fourth direction may be opposite the third direction. In some embodiments, the fourth direction may be substantially perpendicular to the first direction and the second direction. Other configurations are also contemplated. In some embodiments, the second aperture 52 may be disposed between the proximal end of the inflatable orienting element 20 and the distal end of the inflatable orienting element 20. In some embodiments, the second aperture 52 may be disposed between the proximal end of the inflatable orienting element 20 and the distal end of the inflatable orienting element 20 at a location longitudinally offset from and/or longitudinally spaced apart from the first aperture 50. In at least some embodiments, the second aperture 52 may be disposed on an opposite side of the elongate shaft 12 from the first aperture 50.

In some embodiments, the elongate shaft 12 may also include a first radiopaque marker (not shown) located proximate the first aperture 50 to provide an indication of the location of the first aperture 50 under fluoroscopy. In some embodiments, the elongate shaft 12 may include a second radiopaque marker (not shown) located proximate the second aperture 52 to provide an indication of the location of the second aperture 52 under fluoroscopy. Other configurations are also contemplated.

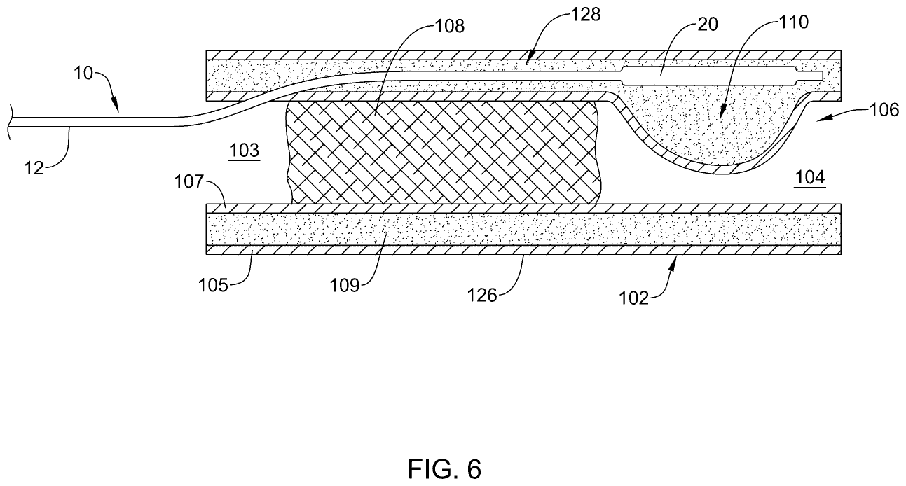

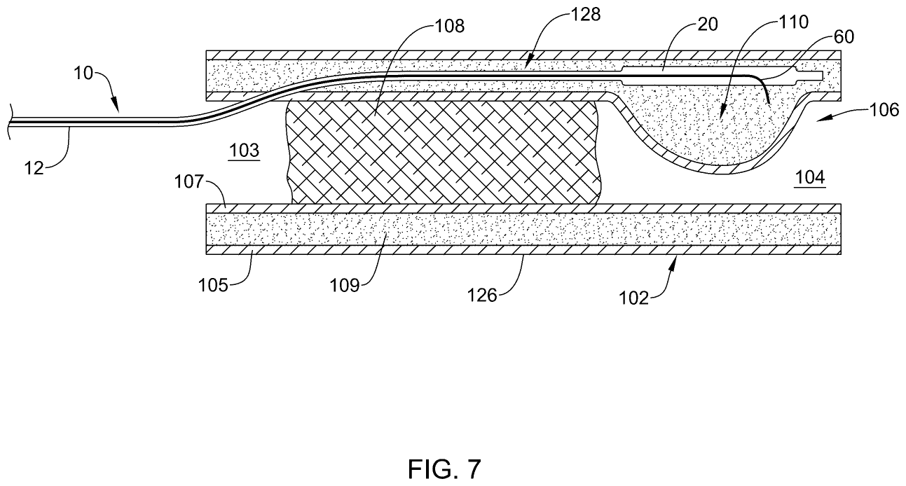

Axial advancement of the distal tip 124 and/or the crossing device 120 within the subintimal space 128 (e.g., FIGS. 1-4) may provide a path for advancement of the self-orienting device 10 into and/or within the subintimal space 128, as seen in FIG. 6. In at least some embodiments, the self-orienting device 10 may be advanced over the guidewire used to place the crossing device 120. The guidewire may be slidably disposed within the working lumen 24 of the self-orienting device 10. The self-orienting device 10 will typically be advanced within the subintimal space 128 to a position distal of the total occlusion 108 before making re-entry into the true lumen 106. As seen in FIG. 6, this may position the self-orienting device 10 adjacent to or within the hematoma 110, if present. The presence of the hematoma 110 may complicate re-entry into the true lumen 106 of the blood vessel 102, and in some cases the presence of the hematoma 110 may be a significant factor in re-entry procedure failure. As shown in FIG. 7, the apparatus may comprise a re-entry device 60 configured to be slidably disposed within and/or advanced within the working lumen 24. The self-orienting device 10 and/or the elongate shaft 12 may be configured to direct the re-entry device 60 toward the true lumen 106 (e.g., the distal segment 104) of the blood vessel 102 at a position distal of the total occlusion 108. In some embodiments, the first aperture 50 and/or the second aperture 52 may be configured to direct the re-entry device 60 toward the true lumen 106 (e.g., the distal segment 104) of the blood vessel 102.

Once positioned in the subintimal space 128, the first inflatable member 20a and the second inflatable member 20b of the inflatable orienting element 20 may be inflated to an expanded/inflated configuration between the innermost layer 107 (e.g., the intima) and the outermost layer 105 (e.g., the adventitia) of the wall 126 (e.g., within the subintimal space 128) of the blood vessel 102. As the first inflatable member 20a and the second inflatable member 20b of the inflatable orienting element 20 are inflated to the expanded/inflated configuration, the first inflatable member 20a and the second inflatable member 20b extend laterally from the elongate shaft 12 within the subintimal space 128 to automatically orient either the first aperture 50 or the second aperture 52 radially inward toward the true lumen 106 of the blood vessel 102. Expansion/inflation of the first inflatable member 20a and the second inflatable member 20b of the inflatable orienting element 20 may rotationally orient the elongate shaft 12 in one of two possible orientations, either with the first aperture 50 facing the true lumen 106 of the blood vessel 102 or with the second aperture 52 facing the true lumen 106 of the blood vessel 102.

However, in some instances where the hematoma 110 is present, the re-entry device 60 may be unable to successfully re-enter the true lumen 106. In some cases, the hematoma 110 may cause the self-orienting device 10 and/or the inflatable orienting element 20 to be misaligned with respect to the true lumen 106. In some cases, the re-entry device 60 may simply extend out of the first aperture 50 or the second aperture 52 into the hematoma 110 instead of through the innermost layer 107 (e.g., the intima) into the true lumen 106 of the blood vessel 102, as shown in FIG. 7.

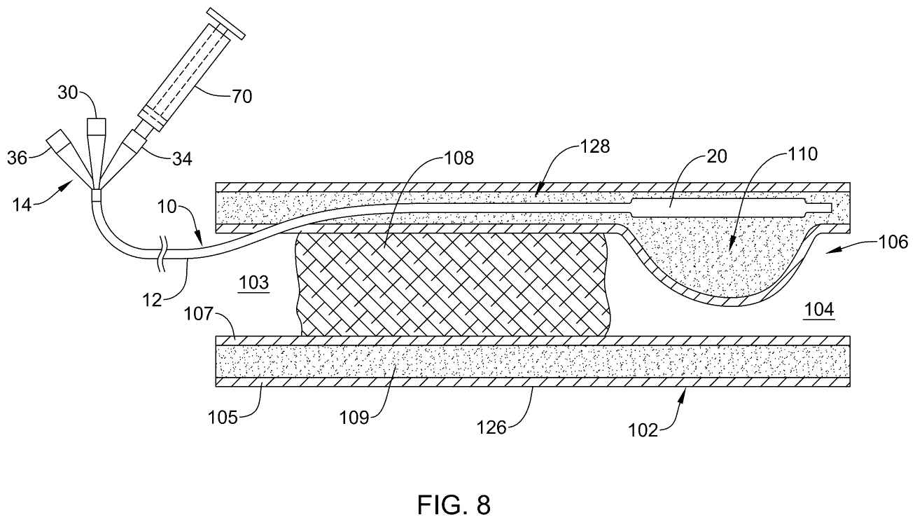

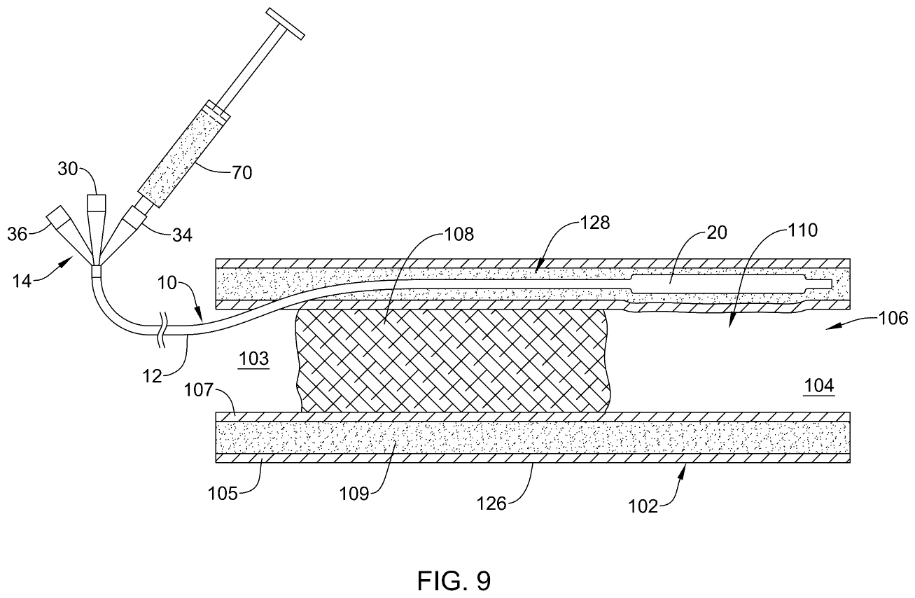

In some embodiments, the apparatus and/or the self-orienting device 10 may be configured to be fluidly connected to a source of suction 70 (e.g., a syringe, a pump, a vacuum source, etc.), as shown in FIG. 8. In some embodiments, the second port 34 may be configured to be fluidly connected to the source of suction 70. Suction may be applied to the working lumen 24 using the source of suction 70 to aspirate blood and/or the hematoma 110 from the subintimal space 128, as shown in FIG. 9. In some embodiments, the distal facing port 28, the first aperture 50, and/or the second aperture 52 may be configured to aspirate blood and/or the hematoma 110 from the subintimal space 128 when the suction is applied to the working lumen 24 using the source of suction 70.

In some embodiments, blood and/or the hematoma 110 may be aspirated from the subintimal space 128 as the self-orienting device 10 and/or the inflatable orienting element 20 is advanced into and/or within the subintimal space 128. In some embodiments, blood may be aspirated as the self-orienting device 10 and/or the inflatable orienting element 20 is advanced into and/or within the subintimal space 128, thereby preventing the hematoma 110 from forming. In some embodiments, blood and/or the hematoma 110 may be aspirated after the self-orienting device 10 and/or the inflatable orienting element 20 is advanced into and/or within the subintimal space 128 and/or after the inflatable orienting element 20 is positioned within the subintimal space 128 at a desired location (e.g., alongside the total occlusion 108, distal of the total occlusion 108, etc.). In some embodiments, blood and/or the hematoma 110 may be aspirated before inflating the inflatable orienting element 20 within the subintimal space 128. In some embodiments, blood and/or the hematoma 110 may be aspirated after inflating the inflatable orienting element 20 within the subintimal space 128. In some embodiments, blood and/or the hematoma 110 may be aspirated while inflating the inflatable orienting element 20 within the subintimal space 128.

In some embodiments, the re-entry device 60 may be disposed within the working lumen 24 prior to advancing the self-orienting device 10 into the subintimal space 128 within the wall 126 of the blood vessel 102 adjacent the total occlusion 108. Accordingly, in some embodiments, blood and/or the hematoma 110 may be aspirated from the subintimal space 128 while the re-entry device 60 is disposed within the working lumen 24. In some embodiments, the re-entry device 60 may be advanced into and/or within the working lumen 24 after aspirating blood and/or the hematoma 110 from the subintimal space 128. As such, in some embodiments, blood and/or the hematoma 110 may be aspirated from the subintimal space 128 before the re-entry device 60 is disposed within the working lumen 24 and/or without the re-entry device 60 disposed within the working lumen 24.

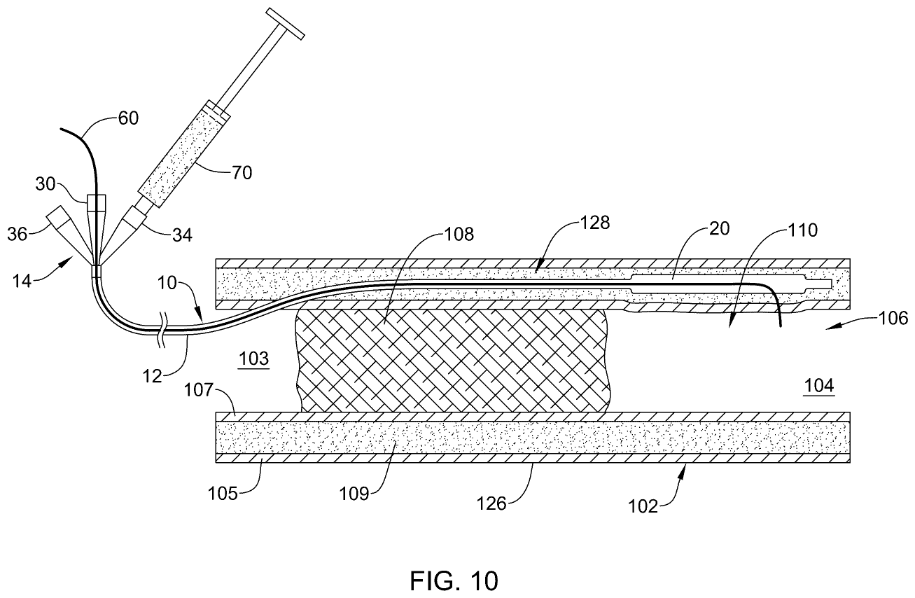

After aspirating blood and/or the hematoma 110 from the subintimal space 128, the re-entry device 60 may be advanced within the working lumen 24 and out the first aperture 50 or the second aperture 52 into the distal segment 104 of the true lumen 106 of the blood vessel 102, as shown in FIG. 10. The re-entry device 60 may be advanced through the working lumen 24 of the self-orienting device 10 and/or the elongate shaft 12 and exit the first aperture 50, if the first aperture 50 is facing the distal segment 104 of the true lumen 106 of the blood vessel 102, to penetrate through the innermost layer 107 (e.g., the intima) into the distal segment 104 of the true lumen 106 of the blood vessel 102 distal of the total occlusion 108. It is noted that if the inflatable orienting element 20 was inflated such that the elongate shaft 12 was oriented with the second aperture 52 facing the distal segment 104 of the true lumen 106 of the blood vessel 102, then the re-entry device 60 may be advanced through the working lumen 24 of the self-orienting device 10 and/or the elongate shaft 12 and exit the second aperture 52 to penetrate through the innermost layer 107 (e.g., the intima) into the distal segment 104 of the true lumen 106 of the blood vessel 102 distal of the total occlusion 108.

In some embodiments, the re-entry device 60 may be the guidewire, or another guidewire introduced through the working lumen 24 of the self-orienting device 10 and/or the elongate shaft 12. In some instances, the re-entry device 60 may have a pre-formed bent distal end portion in which the distal end portion is bent at an oblique angle to a proximal portion of the re-entry device 60. The bent distal end portion may facilitate exiting the first aperture 50 or the second aperture 52 when the distal tip of the re-entry device 60 encounters the first aperture 50 or the second aperture 52. In other instances, the self-orienting device 10 and/or the elongate shaft 12 may include a deflection mechanism to deflect the re-entry device 60 out through the first aperture 50 or the second aperture 52. In some instances, the re-entry device 60 may include a reduced diameter penetrating tip to facilitate penetrating through the innermost layer 107 (e.g., the intima). In other embodiments, the re-entry device 60 may be an elongate member, such as a needle cannula or stylet, having a sharpened distal tip configured to pierce through the innermost layer 107 (e.g., the intima) into the distal segment 104 of the true lumen 106 of the blood vessel 102 distal of the total occlusion 108.

In some instances, fluoroscopy may be utilized to confirm the trajectory of the re-entry device 60 from the self-orienting device 10 and/or the elongate shaft 12 to ensure the re-entry device 60 is being advanced toward the distal segment 104 of the true lumen 106 and not radially outward through the outermost layer 105 (e.g., the adventitia).

In some embodiments, a guidewire (which may be the same guidewire used to place the self-orienting device 10, or may be a different guidewire) may be advanced within a lumen extending through the re-entry device 60 into the distal segment 104 of the true lumen 106 of the blood vessel 102. Subsequently, one or more additional medical devices may be advanced over the guidewire from the proximal segment 103 of the true lumen 106 of the blood vessel 102, through the subintimal space 128 within the wall 126 of the blood vessel 102 alongside and/or adjacent to the total occlusion 108, and into the distal segment 104 of the true lumen 106 of the blood vessel 102.

In some instances, a trapping balloon catheter may be used to maintain the position of the re-entry device 60 (or a subsequently placed guidewire) through the subintimal space 128 around the total occlusion 108 while the self-orienting device 10 is withdrawn, a process known as a trapping technique. For instance, the self-orienting device 10 may have been advanced through a guide catheter (not shown), such as a 6F, 7F or 8F guide catheter, to reach the subintimal space 128 alongside the total occlusion 108. Prior to withdrawing the self-orienting device 10 from the blood vessel 102 subsequent to successful re-entry into the distal segment 104 of the true lumen 106 distal of the total occlusion 108, a trapping balloon catheter may be advanced through the guide catheter alongside an exterior of the self-orienting device 10 and then the trapping balloon of the trapping balloon catheter may be inflated distal of the self-orienting device 10 within the guide catheter to secure the re-entry device 60 or other guidewire against the inner wall of the guide catheter. With the re-entry device 60 or other guidewire firmly secured to the inner wall of the guide catheter to prevent longitudinal movement thereof, the self-orienting device 10 may be withdrawn from the guide catheter and the blood vessel 102. Once the self-orienting device 10 is withdrawn, the trapping balloon of the trapping balloon catheter may be deflated, leaving the re-entry device 60 or another guidewire in place.

Once a pathway has been created across the total occlusion 108, (e.g., around the total occlusion 108 via the subintimal space 128), one or more additional medical devices may be advanced through the blood vessel 102 over the re-entry device 60 or other guidewire to enlarge the pathway and/or pass distally of the total occlusion 108 to perform a further medical procedure.

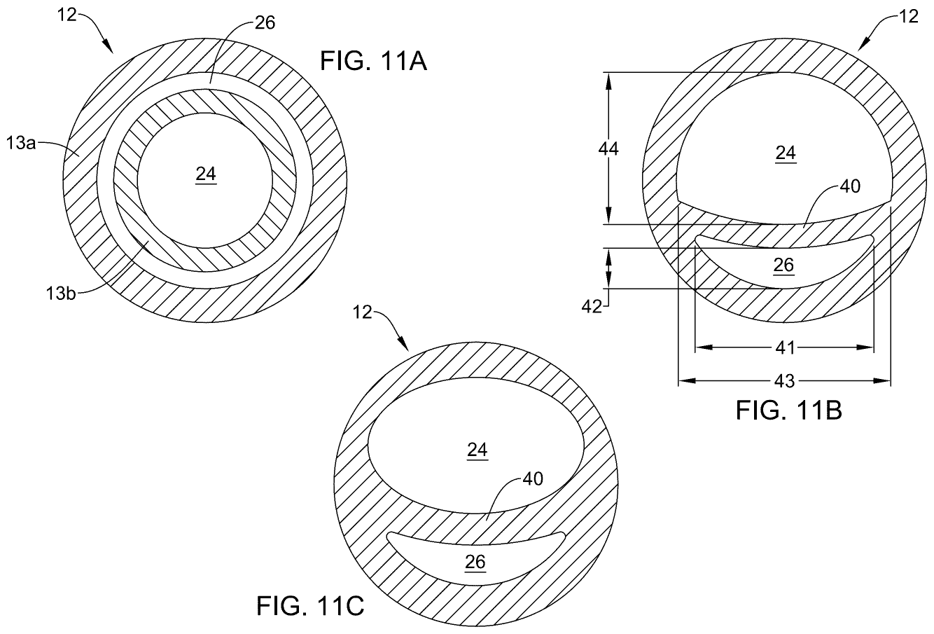

FIGS. 11A and 11B are cross-sectional views taken along the line 11-11 in FIG. 5 of a proximal portion of the elongate shaft 12 of the self-orienting device 10. In some embodiments, an outer diameter of the proximal portion of the elongate shaft 12 may be about 0.042 inches or less, which would enable the trapping technique in a 6F or larger guide catheter. In some embodiments, the proximal portion of the elongate shaft 12 may have an outer wall thickness of about 0.005 inches. Other configurations are also contemplated. When attempting to aspirate through the working lumen 24, certain parameters may be considered desirable. For example, it may be desirable and/or a design goal to maximize aspiration rate through the working lumen 24 while maintaining a deflation time for the inflatable orienting element 20 of less than 10 seconds (a time considered acceptable to users). The deflation time and/or aspiration rate may be determined and/or defined by the size of the structures of the elongate shaft 12.

FIG. 11A illustrates a tube-in-tube dual lumen shaft. The elongate shaft 12 may include an outer tube 13a and an inner tube 13b disposed within the outer tube 13a. The working lumen 24 may be disposed within the inner tube 13b. The inflation lumen 26 may be disposed between the inner tube 13b and the outer tube 13a. Both the inner tube 13b and the outer tube 13a may have circular cross-sectional shapes. Accordingly, the inflation lumen 26 may be annular in shape. In some embodiments, the outer tube 13a may have an outer diameter of about 0.042 inches and an inner diameter of about 0.032 inches. In some embodiments, the inner tube 13b may have an outer diameter of about 0.023 inches and an inner diameter of about 0.017 inches. The outer tube 13a may have a wall thickness of about 0.005 inches and the inner tube 13b may have a wall thickness of about 0.003 inches.

The configuration described above may be used as and/or considered to be a baseline for determining aspiration rate and deflation time. In order to maintain structural integrity of the elongate shaft 12, and for operational reasons including but not limited to compatibility with other medical devices used in the relevant procedures, the outer diameter of the outer tube 13a (e.g., 0.042 inches), which enables the trapping technique in a 6F or larger guide catheter, and the wall thickness of the outer tube 13a (e.g., 0.005 inches) will be maintained. The inner diameter and/or the outer diameter of the inner tube 13b were varied to determine the effect on aspiration rate and deflation time. Through calculation and/or bench testing, the following aspiration rates and deflation times have been determined for varying sizes of the inner tube 13b. For reference, aspiration rates were tested (and calculated, with a correspondence appearing therebetween) while deflation times were calculated based on the size of the lumen. The formula used in the calculation for aspiration rates (of the working lumen 24, which is circular) is similar to or a rearrangement of the Darcy-Weisbach equation using volumetric flow rate instead of fluid velocity:

$$Q = ( πΔ P / 8 μ L ) {( r )}^{4}$$

For the purpose of calculation, π, ΔP, and μ are constants, and L (length) was also maintained at a constant value. The variable “r” corresponds to the radius of the circular lumen (e.g., the radius of the working lumen 24). The formula was correlated with the aspiration rate by comparing the calculated aspiration value with the measured aspiration value.

Calculated deflation times are found using the calculated volumetric flow rate for the inflation lumen 26, which is annular. The formula for flow rate in an annular lumen is:

$$Q = ( πΔ P / 8 μ L ) ( {a}^{4} - {b}^{4} - ( {( {a}^{2} - {b}^{2} )}^{2} / ln ( a / b ) ) )$$

For the purpose of calculation, π, ΔP, and μ are constants, and L was also maintained at a constant value. The variable “a” corresponds to the major diameter of the annular lumen and the variable “b” corresponds to the minor diameter of the annular lumen. Using this formula, the calculated deflation times are believed to be reasonably accurate and representative of actual values for the purpose of comparison.

| Inner | Outer | Inner | Aspiration | Deflation | |
|---|---|---|---|---|---|
| Tube | Diameter | Diameter | Rate | Time | |
| Baseline | 0.0230 | 0.0170 | 1x | 1.5 | sec |
| A | 0.0270 | 0.0200 | 1.9x | 8.4 | sec |
| B | 0.0280 | 0.0207 | 2.2x | 16.1 | sec |
| C | 0.0290 | 0.0214 | 2.5x | 37 | sec |
| D | 0.0300 | 0.0222 | 2.9x | 124 | sec |

In view of the above data, applicants determined that forming the inner tube 13b to have configuration A, with an outer diameter of 0.0270 inches and an inner diameter of 0.0200 inches produced the highest aspiration rate while maintaining the deflation time below the 10 second target using the tube-in-tube design. The cross-sectional area of the working lumen 24 in configuration A is about 0.00031415 square inches, and the cross-sectional area of the inflation lumen 26 in configuration A is about 0.00023169 square inches.

By switching to a dual-lumen design within a single tube or shaft, applicants found that the aspiration rate may be increased even further while maintaining the deflation time below the 10 second target. FIG. 11B illustrates a dual-lumen shaft formed from a single extrusion. In some embodiments, in the proximal portion of the elongate shaft 12 proximal of the inflatable orienting element 20, the inflation lumen 26 may have a first noncircular cross-sectional shape and the working lumen 24 may have a second noncircular cross-sectional shape. In some embodiments, the first noncircular cross-sectional shape may be crescent shaped. Similar to tube-in-tube design described above, an outer diameter of the proximal portion of the elongate shaft 12 is maintained at 0.042 inches, which enables the trapping technique in a 6F or larger guide catheter, and an outer wall thickness is maintained at 0.005 inches for the sake of comparison.

In one example configuration of the proximal portion of the elongate shaft 12 shown in FIG. 11B, the elongate shaft 12 may have an internal web 40 separating the working lumen 24 from the inflation lumen 26. The internal web 40 may have a thickness of about 0.003 inches. The first noncircular cross-sectional shape of the inflation lumen 26 may have a major dimension 41 of about 0.027 inches and a minor dimension 42 of about 0.006 inches. The second noncircular cross-sectional shape of the working lumen 24 may have a first maximum extent 43 extending parallel to the major dimension 41 of the first noncircular cross-sectional shape of the inflation lumen 26, and a second maximum extent 44 oriented perpendicular to the first maximum extent 43 and/or the major dimension 41 of the first noncircular cross-sectional shape of the inflation lumen 26. In at least some embodiments, the second maximum extent 44 is less than the first maximum extent 43. In some embodiments, the first maximum extent 43 may be about 0.032 inches. In some embodiments, the first maximum extent 43 is greater than the major dimension 41. In some embodiments, the second maximum extent may be about 0.023 inches.

Using the above dimensions for the first noncircular cross-sectional shape of the inflation lumen 26 and the second noncircular cross-sectional shape of the working lumen 24, the cross-sectional area of the first noncircular cross-sectional shape and/or the inflation lumen 26 may be about 0.000134 square inches, and the cross-sectional area of the second noncircular cross-sectional shape and/or the working lumen 24 may be about 0.000565 square inches. In some instances, the cross-sectional area of the first noncircular cross-sectional shape and/or the inflation lumen 26 may be about 0.00013 square inches or more, and the cross-sectional area of the second noncircular cross-sectional shape and/or the working lumen 24 may be about 0.00056 square inches or more. The first noncircular cross-sectional shape of the inflation lumen 26 may have a perimeter length of about 0.06309 inches. The second noncircular cross-sectional shape of the working lumen 24 may have a perimeter length of about 0.086953 inches. In some instances, the perimeter of the first noncircular cross-sectional shape and/or the inflation lumen 26 may be about 0.063 inches or less, and the perimeter of the second noncircular cross-sectional shape and/or the working lumen 24 may be about 0.087 inches or less.

Using the above dimensions, the aspiration rate using the second noncircular cross-sectional shape and/or the working lumen 24 is about 5.5× the aspiration rate of the baseline configuration of the tube-in-tube design of FIG. 11A above and the deflation time using the first noncircular cross-sectional shape and/or the inflation lumen 26 is 7.9 seconds. This can be compared to the 1.9× aspiration rate and 8.4 second deflation time achieved using configuration A of the tube-in-tube design of FIG. 11A. The dual-lumen design of FIG. 11B produced a significant improvement in aspiration rate while maintaining the deflation time below the 10 second target. Additionally, in one test, the second noncircular cross-sectional shape of the working lumen 24 of FIG. 11B aspirated on average about 6× more fluid by mass than the baseline configuration of the tube-in-tube design of FIG. 11A over a 20 second test period using a reference fluid of 40% glycerol and 60% water, whereas configuration A of the tube-in-tube design of FIG. 11A aspirated on average about 1.5× more fluid by mass than the baseline configuration of the tube-in-tube design of FIG. 11A over the 20 second test period using the reference fluid of 40% glycerol and 60% water.

The formula used in the calculation of aspiration rates and deflation times in noncircular lumens (e.g., the lumens in the dual-lumen design of FIG. 11B) is:

Q=(πΔP/8 μL)(2A/P)⁴

For the purpose of calculation, π, ΔP, and μ are constants, and L was also maintained at a constant value. The variable A corresponds to the cross-sectional area of the lumen and the variable P corresponds to the perimeter length of the lumen (e.g., a perimeter length around the cross-sectional area), such that (2A/P) corresponds to the size of the lumen. As an example, if the lumen is round/circular, (2A/P) reduces down to equal the radius of the lumen, thereby confirming the relationship between the equations used herein. The variable (2A/P) is suitable for noncircular and/or irregularly shaped lumens, as well as circular and/or regularly shaped lumens.

The improvement in aspiration rate seen in the dual-lumen design over the tube-in-tube design comes from a more efficient use of space within the elongate shaft 12. Based on volumetric flow calculations using the above-referenced formula, a relevant factor in transferring more mass is a ratio of lumen cross-sectional area to lumen perimeter of the lumen, wherein maximizing the ratio of lumen cross-sectional area to lumen perimeter increases volumetric flow (e.g., increasing lumen cross-sectional area while decreasing lumen perimeter, maintaining lumen cross-sectional area while decreasing lumen perimeter, increasing lumen cross-sectional area while maintaining lumen perimeter, etc.).

In some embodiments, the outer diameter of the proximal portion of the elongate shaft 12 may be 0.045 inches or less, the cross-sectional area of the first noncircular cross-sectional shape and/or the inflation lumen 26 may be at least 0.00013 square inches and the perimeter of the first noncircular cross-sectional shape and/or the inflation lumen 26 may be 0.063 inches or less, and the cross-sectional area of the second noncircular cross-sectional shape and/or the working lumen 24 may be at least 0.00056 square inches and the perimeter of the second noncircular cross-sectional shape and/or the working lumen 24 may be 0.087 inches or less. In some embodiments, the outer diameter of the proximal portion of the elongate shaft 12 may be 0.042 inches or less, the cross-sectional area of the first noncircular cross-sectional shape and/or the inflation lumen 26 may be at least 0.000134 square inches and the perimeter of the first noncircular cross-sectional shape and/or the inflation lumen 26 may be 0.06309 inches or less, and the cross-sectional area of the second noncircular cross-sectional shape and/or the working lumen 24 may be at least 0.000565 square inches and the perimeter of the second noncircular cross-sectional shape and/or the working lumen 24 may be 0.086953 inches or less. Other dimensions and/or configurations are also contemplated that may satisfy the desired parameters.

FIG. 11C illustrates selected aspects of an alternative configuration of the dual lumen design of FIG. 11B. In some embodiments, in the proximal portion of the elongate shaft 12 proximal of the inflatable orienting element 20, the inflation lumen 26 may have a first noncircular cross-sectional shape and the working lumen 24 may have a second noncircular cross-sectional shape. In some embodiments, the first noncircular cross-sectional shape may be crescent shaped. In some embodiments, the second noncircular cross-sectional shape may be elliptical. Similar to designs described above, an outer diameter of the proximal portion of the elongate shaft 12 may be maintained at 0.042 inches, which enables the trapping technique in a 6F or larger guide catheter. In the dual lumen design illustrated in FIG. 11C, the outer wall thickness is increased to 0.006 inches and the internal web has a thickness of about 0.004 inches. In the dual lumen design of FIG. 11C, the thickness of the wall of the elongate shaft 12 and the thickness of the internal web have been increased slightly to consider the aspiration rate and deflation time where the thicknesses were increased for additional column strength, kink resistance, and/or other structural characteristics.

The first noncircular cross-sectional shape of the inflation lumen 26 may have a major dimension of about 0.025 inches and a minor dimension of about 0.006 inches. The second noncircular cross-sectional shape of the working lumen 24 may have a major dimension extending parallel to the major dimension of the first noncircular cross-sectional shape of the inflation lumen 26, and a minor dimension oriented perpendicular to the major dimension and/or perpendicular to the major dimension of the first noncircular cross-sectional shape of the inflation lumen 26. In some embodiments, the minor dimension of the second noncircular cross-sectional shape of the working lumen 24 may be parallel to the minor dimension of the first noncircular cross-sectional shape of the inflation lumen 26. In at least some embodiments, the major dimension of the first noncircular cross-sectional shape of the inflation lumen 26 is less than the major dimension of the second noncircular cross-sectional shape of the working lumen 24. In some embodiments, the major dimension of the second noncircular cross-sectional shape of the working lumen 24 may be about 0.029 inches. In some embodiments, the major dimension of the second noncircular cross-sectional shape of the working lumen 24 is greater than the major dimension of the first noncircular cross-sectional shape of the inflation lumen 26. In some embodiments, the minor dimension of the second noncircular cross-sectional shape of the working lumen 24 may be about 0.020 inches.

Using the above dimensions for the first noncircular cross-sectional shape of the inflation lumen 26 and the second noncircular cross-sectional shape of the working lumen 24, the cross-sectional area of the first noncircular cross-sectional shape and/or the inflation lumen 26 may be about 0.000125 square inches, and the cross-sectional area of the second noncircular cross-sectional shape and/or the working lumen 24 may be about 0.000455 square inches. In some instances, the cross-sectional area of the first noncircular cross-sectional shape and/or the inflation lumen 26 may be about 0.0001 square inches or more, and the cross-sectional area of the second noncircular cross-sectional shape and/or the working lumen 24 may be about 0.0004 square inches or more. The first noncircular cross-sectional shape of the inflation lumen 26 may have a perimeter length of about 0.06412 inches. The second noncircular cross-sectional shape of the working lumen 24 may have a perimeter length of about 0.07762 inches. In some instances, the perimeter of the first noncircular cross-sectional shape and/or the inflation lumen 26 may be about 0.065 inches or less, and the perimeter of the second noncircular cross-sectional shape and/or the working lumen 24 may be about 0.08 inches or less.

Using the above dimensions, the aspiration rate using the second noncircular cross-sectional shape and/or the working lumen 24 is about 3.6× the aspiration rate of the baseline configuration of the tube-in-tube design of FIG. 11A above and the deflation time using the first noncircular cross-sectional shape and/or the inflation lumen 26 is 11.1 seconds. This can be compared to the 1.9× aspiration rate and 8.4 second deflation time achieved using configuration A of the tube-in-tube design of FIG. 11A. The dual lumen design of FIG. 11C produced a nearly two-fold improvement in aspiration rate while providing a deflation time about 10% above the 10 second target. In some embodiments, these values may be considered acceptable if the increased wall thickness is needed.

The formula used in the calculation of aspiration rates and deflation times in noncircular lumens (e.g., the lumens in the dual-lumen design of FIG. 11C) is:

$$Q = ( πΔ P / 8 μ L ) {( 2 A / P )}^{4}$$

For the purpose of calculation, π, ΔP, and μ are constants, and L was also maintained at a constant value. The variable A corresponds to the cross-sectional area of the lumen and the variable P corresponds to the perimeter length of the lumen (e.g., a perimeter length around the cross-sectional area), such that (2A/P) corresponds to the size of the lumen. As an example, if the lumen is round/circular, (2A/P) reduces down to equal the radius of the lumen, thereby confirming the relationship between the equations used herein. The variable (2A/P) is suitable for noncircular and/or irregularly shaped lumens, as well as circular and/or regularly shaped lumens.

In some embodiments, the outer diameter of the proximal portion of the elongate shaft 12 may be 0.045 inches or less, the cross-sectional area of the first noncircular cross-sectional shape and/or the inflation lumen 26 may be at least 0.00012 square inches and the perimeter of the first noncircular cross-sectional shape and/or the inflation lumen 26 may be 0.065 inches or less, and the cross-sectional area of the second noncircular cross-sectional shape and/or the working lumen 24 may be at least 0.00045 square inches and the perimeter of the second noncircular cross-sectional shape and/or the working lumen 24 may be 0.078 inches or less. In some embodiments, the outer diameter of the proximal portion of the elongate shaft 12 may be 0.042 inches or less, the cross-sectional area of the first noncircular cross-sectional shape and/or the inflation lumen 26 may be at least 0.000125 square inches and the perimeter of the first noncircular cross-sectional shape and/or the inflation lumen 26 may be 0.06412 inches or less, and the cross-sectional area of the second noncircular cross-sectional shape and/or the working lumen 24 may be at least 0.000456 square inches and the perimeter of the second noncircular cross-sectional shape and/or the working lumen 24 may be 0.07762 inches or less. Other dimensions and/or configurations are also contemplated that may satisfy the desired parameters.

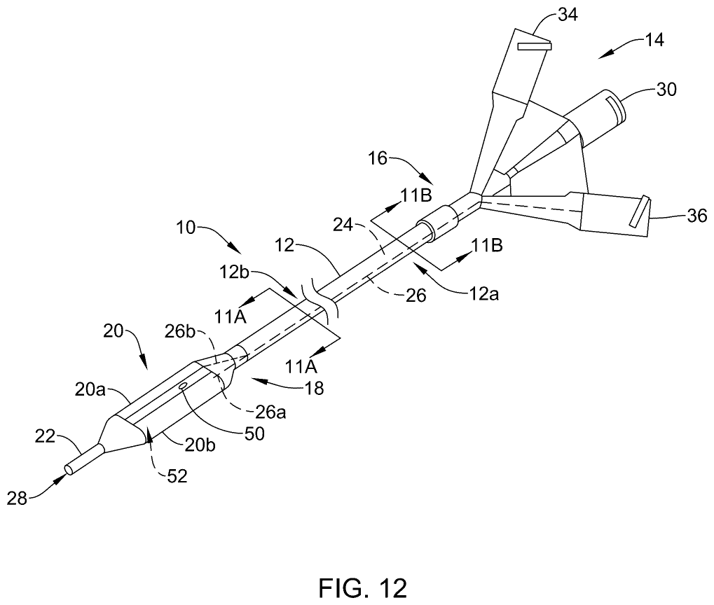

In at least some embodiments, the proximal portion of the elongate shaft 12 proximal of the inflatable orienting element 20 may comprise a proximal section 12a having the dual lumen design of FIG. 11B and a distal section 12b having the tube-in-tube design of FIG. 11A, as seen in FIG. 12. In some embodiments, the proximal portion of the elongate shaft 12 may comprise the proximal section 12a wherein the inflation lumen 26 has the first noncircular cross-sectional shape and the working lumen 24 has the second noncircular cross-sectional shape, and the proximal portion of the elongate shaft 12 may comprise the distal section 12b wherein the inflation lumen 26 and the working lumen 24 each have circular cross-sectional shapes. In some embodiments, in the proximal section 12a, the inflation lumen 26 and the working lumen 24 are oriented noncoaxially, side-by-side, and/or parallel with each other. In some embodiments, in the distal section 12b, the inflation lumen 26 and the working lumen 24 are oriented coaxially with each other. Other configurations are also contemplated. In some embodiments, the configuration shown in FIG. 12 may be considered a hybrid configuration. When considering the formula used in the calculation of aspiration rates and deflation times in noncircular lumens, it may be seen that length may be a major factor in determining flow characteristics. In at least some instances, length may dominate the flow characteristics.

In some embodiments, the length of the proximal section 12a of the elongate shaft 12 may be about 50% of the overall length of the proximal portion of the elongate shaft 12. In some embodiments, the length of the proximal section 12a of the elongate shaft 12 may be at least 50% of the overall length of the proximal portion of the elongate shaft 12. In some embodiments, the proximal section 12a of the elongate shaft 12 may be longer than the distal section 12b of the elongate shaft 12. In some embodiments, the length of the proximal section 12a of the elongate shaft 12 may be about 60% of the overall length of the proximal portion of the elongate shaft 12 and the length of the distal section 12b of the elongate shaft 12 may be about 40% over the overall length of the proximal portion of the elongate shaft 12. In some embodiments, the length of the proximal section 12a of the elongate shaft 12 may be at least 60% of the overall length of the proximal portion of the elongate shaft 12. In some embodiments, the length of the proximal section 12a of the elongate shaft 12 may be about 65% of the overall length of the proximal portion of the elongate shaft 12 and the length of the distal section 12b of the elongate shaft 12 may be about 35% over the overall length of the proximal portion of the elongate shaft 12. In some embodiments, the length of the proximal section 12a of the elongate shaft 12 may be at least 65% of the overall length of the proximal portion of the elongate shaft 12. In some embodiments, the length of the proximal section 12a of the elongate shaft 12 may be about 70% of the overall length of the proximal portion of the elongate shaft 12 and the length of the distal section 12b of the elongate shaft 12 may be about 30% over the overall length of the proximal portion of the elongate shaft 12. In some embodiments, the length of the proximal section 12a of the elongate shaft 12 may be at least 70% of the overall length of the proximal portion of the elongate shaft 12. In some embodiments, the length of the proximal section 12a of the elongate shaft 12 may be about 75% of the overall length of the proximal portion of the elongate shaft 12 and the length of the distal section 12b of the elongate shaft 12 may be about 25% over the overall length of the proximal portion of the elongate shaft 12. In some embodiments, the length of the proximal section 12a of the elongate shaft 12 may be at least 75% of the overall length of the proximal portion of the elongate shaft 12. In some embodiments, the length of the proximal section 12a of the elongate shaft 12 may be about 80% of the overall length of the proximal portion of the elongate shaft 12 and the length of the distal section 12b of the elongate shaft 12 may be about 20% over the overall length of the proximal portion of the elongate shaft 12. In some embodiments, the length of the proximal section 12a of the elongate shaft 12 may be at least 80% of the overall length of the proximal portion of the elongate shaft 12. In some embodiments, the length of the proximal section 12a of the elongate shaft 12 may be about 85% of the overall length of the proximal portion of the elongate shaft 12 and the length of the distal section 12b of the elongate shaft 12 may be about 15% over the overall length of the proximal portion of the elongate shaft 12. In some embodiments, the length of the proximal section 12a of the elongate shaft 12 may be at least 85% of the overall length of the proximal portion of the elongate shaft 12. In some embodiments, the length of the proximal section 12a of the elongate shaft 12 may be about 90% of the overall length of the proximal portion of the elongate shaft 12 and the length of the distal section 12b of the elongate shaft 12 may be about 10% over the overall length of the proximal portion of the elongate shaft 12. In some embodiments, the length of the proximal section 12a of the elongate shaft 12 may be at least 90% of the overall length of the proximal portion of the elongate shaft 12. In some embodiments, the length of the proximal section 12a of the elongate shaft 12 may be about 95% of the overall length of the proximal portion of the elongate shaft 12 and the length of the distal section 12b of the elongate shaft 12 may be about 5% over the overall length of the proximal portion of the elongate shaft 12. In some embodiments, the length of the proximal section 12a of the elongate shaft 12 may be at least 95% of the overall length of the proximal portion of the elongate shaft 12. Other configurations are also contemplated.

While the tube-in-tube design of FIG. 11A exhibits lower aspiration rates and slower inflation times compared to the dual lumen design of FIG. 11B, in the hybrid configuration of FIG. 12 the values will be proportional to length. Accordingly, the aspirate rate(s) and the inflation time(s) associated with the configuration of FIG. 12 may see the increased aspiration rate(s) and faster inflation time(s) associated with the dual lumen design of FIG. 11B along a majority of the length of the proximal portion of the elongate shaft 12, thereby achieving a similar percentage of the benefit as a percentage of the overall length of the proximal portion of the elongate shaft 12 that is formed using the dual lumen design of FIG. 11B. For example, if the overall length of the proximal portion of the elongate shaft 12 is formed as about 80% dual lumen design and about 20% tube-in-tube design, the aspiration rate would be about 80% of the aspiration rate achieved if the entire proximal portion of the elongate shaft 12 was formed using the dual lumen design. As such, the hybrid configuration of FIG. 12 may still achieve significant improvement over the tube-in-tube design of FIG. 11A.

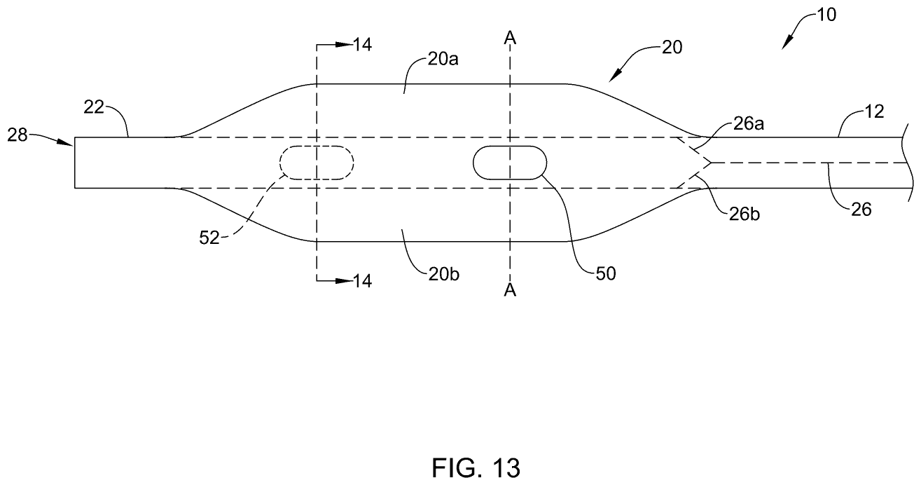

FIGS. 13-15 illustrate selected aspects of the self-orienting device 10 of FIG. 5 in greater detail. Elements and/or features of the self-orienting device 10 that are not expressly discussed with respect to FIGS. 13-15 may be discussed with respect to FIG. 5, and vice versa. In some embodiments, the inflatable orienting element 20 may be disposed on and/or mounted on the elongate shaft 12. In FIG. 13, the inflatable orienting element 20 is shown in an inflated configuration. In shall be understood that the inflatable orienting element 20 may assume and/or may be constrained in a collapsed configuration and/or an uninflated configuration for delivery and/or advancement into the subintimal space 128 (e.g., FIG. 6). The inflatable orienting element 20 may be configured to shift from the collapsed configuration and/or the uninflated configuration toward and/or to the inflated configuration after the inflatable orienting element 20 has been positioned at a desired location within the subintimal space 128. In some embodiments, the elongate shaft 12 may extend through the inflatable orienting element 20 and/or the elongate shaft 12 may extend distal of the inflatable orienting element 20. In some embodiments, the elongate shaft 12 may include the distal tip 22 extending distal of the inflatable orienting element 20.

In some embodiments, the inflatable orienting element 20 may comprise the first inflatable member 20a and the second inflatable member 20b, as described herein. In some embodiments, the inflatable orienting element 20, the first inflatable member 20a, and/or the second inflatable member 20b may be formed from extruded portions of an outer wall of the elongate shaft 12. In some embodiments, the outer wall of the elongate shaft 12 may define and/or include the first aperture 50 and/or the second aperture 52. In some embodiments, the first aperture 50 and/or the second aperture 52 may be disposed laterally between the first inflatable member 20a and the second inflatable member 20b.

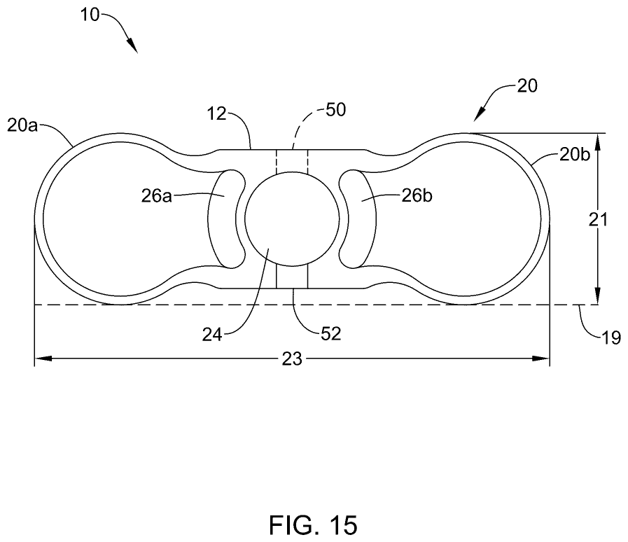

FIG. 13 schematically illustrates a path of the inflation lumen 26 within the elongate shaft 12. As seen in FIG. 13, as the inflation lumen 26 approaches the inflatable orienting element 20, the inflation lumen 26 may split (e.g., bifurcate) into a first inflation lumen 26a in fluid communication with the first inflatable member 20a and a second inflation lumen 26b in fluid communication with the second inflatable member 20b. See also FIG. 15. In some embodiments, the inflation lumen 26 may form a Y-junction with the first inflation lumen 26a and the second inflation lumen 26b within the elongate shaft 12 and/or within the inflatable orienting element 20. For example, a distal portion of the elongate shaft 12 may include the working lumen 24, the inflation lumen 26 (shown as a bifurcated lumen including a first inflation lumen 26a and a second inflation lumen 26b. In some instances, the second inflation lumen 26b may extend parallel to and spaced apart from the first inflation lumen 26a. The cross-sectional shape of the elongate shaft 12 in the distal portion of the elongate shaft 12 may be different than the cross-sectional shape of the elongate shaft 12 in the proximal portion of the elongate shaft 12. For example, the proximal portion of the elongate shaft 12 may have a circular cross-sectional shape, whereas the distal portion of the elongate shaft 12 may have a non-circular cross-sectional shape, such as a generally rectangular cross-sectional shape.

The position and arrangement of the various lumens in the proximal portion of the elongate shaft 12 may be dissimilar from the position and arrangement of the lumens in the distal portion of the elongate shaft 12. For example, in the distal portion of the elongate shaft 12 the first and second inflation lumens 26a, 26b may be positioned on opposite sides of the guidewire lumen 24, with the guidewire lumen 24 positioned between the first and second inflation lumens 26a, 26b. Other configurations are also contemplated.

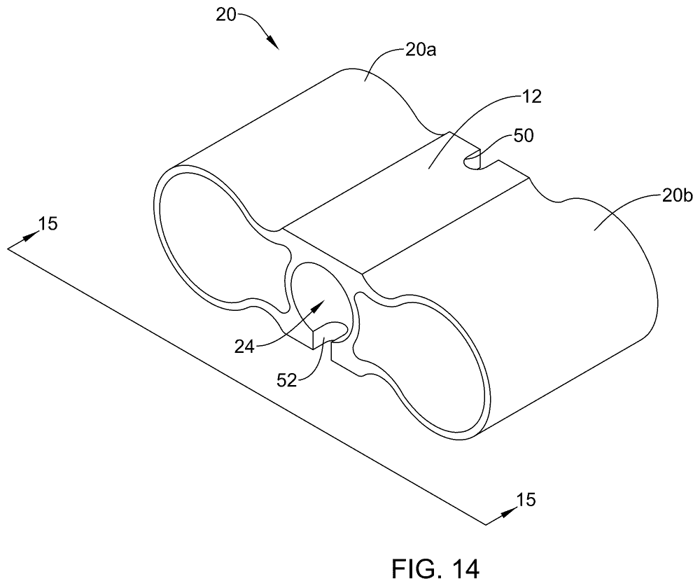

FIG. 14 is a perspective view of a portion of the inflatable orienting element 20, wherein cuts are made at line 14-14 and at line A-A on FIG. 13. In some embodiments, the first inflatable member 20a may extend laterally from the central longitudinal axis of the elongate shaft 12 in a first direction, and the second inflatable member 20b may extend laterally from the central longitudinal axis of the elongate shaft 12 in a second direction different from the first direction. In at least some embodiments, the second direction may be opposite the first direction. The first aperture 50 may open in a third direction different from the first direction and the second direction. In some embodiments, the third direction may be perpendicular to the first direction and the second direction. The second aperture 52 may open in a fourth direction different from the third direction. In some embodiments, the fourth direction may be opposite the third direction. In some embodiments, the fourth direction may be perpendicular to the first direction and/or the second direction. Other configurations are also contemplated.

FIG. 15 is a cross-sectional view taken along the line 15-15 in FIG. 14 illustrating selected aspects of the self-orienting device 10. As seen in FIG. 15, the working lumen 24 may extend through the inflatable orienting element 20 between the first inflatable member 20a and the second inflatable member 20b. While illustrated in FIG. 15 as having a circular cross-sectional shape, the working lumen 24 may have the second noncircular cross-sectional shape described herein. Additionally, while the working lumen 24 is shown in FIG. 15 as being generally coaxial with the central longitudinal axis of the elongate shaft 12 within the inflatable orienting element 20, this arrangement is not required, and the working lumen 24 may be radially offset from the central longitudinal axis of the elongate shaft 12, as shown in FIGS. 11B-11C. FIG. 15 also illustrates the first inflation lumen 26a in fluid communication with the first inflatable member 20a and the second inflation lumen 26b in fluid communication with the second inflatable member 20b. In some embodiments, the first inflation lumen 26a and the second inflation lumen 26b may be positioned in a generally planetary arrangement around the working lumen 24, but this arrangement is not required. Other positioning, shapes, and/or arrangements for the first inflation lumen 26a and/or the second inflation lumen 26b are also contemplated.

In at least some embodiments, the first inflation lumen 26a and/or the second inflation lumen 26b may terminate at a proximal end of the inflatable orienting element 20, the first inflatable member 20a, and/or the second inflatable member 20b. In some embodiments, the first inflation lumen 26a and/or the second inflation lumen 26b may extend into a body of the inflatable orienting element 20, the first inflatable member 20a, and/or the second inflatable member 20b. In some embodiments, the first inflation lumen 26a and/or the second inflation lumen 26b may terminate proximal of the first aperture 50 and/or the second aperture 52.

In some embodiments, the inflation lumen 26 and/or the first noncircular cross-sectional shape may terminate proximal to the inflatable orienting element 20. In some embodiments, the first inflation lumen 26a and the second inflation lumen 26b may extend distally from a distal end and/or a termination point of the inflation lumen 26 and/or the first noncircular cross-sectional shape. In some embodiments, the first inflation lumen 26a and/or the second inflation lumen 26b may shift position within the elongate shaft 12 (e.g., may move from a “bottom” of the elongate shaft 12, as seen in FIGS. 11B-C, to “sides” of the elongate shaft 12, as seen in FIG. 15) such that the first aperture 50 and/or the second aperture 52 may connect to and/or communicate with the working lumen 24 without crossing, penetrating, or interfering with the inflation lumen 26, the first inflation lumen 26a, and/or the second inflation lumen 26b. Other configurations are also contemplated.

Returning to FIG. 15, in some embodiments, the first inflatable member 20a may be monolithically and/or seamlessly formed with the elongate shaft 12. In some embodiments, the first inflatable member 20a may comprise an extruded portion of the outer wall of the elongate shaft 12. In some embodiments, the second inflatable member 20b may be monolithically and/or seamlessly formed with the elongate shaft 12. In some embodiments, the second inflatable member 20b may comprise an extruded portion of the outer wall of the elongate shaft 12.

In some embodiments, the first inflatable member 20a and the second inflatable member 20b may assume a generally planar arrangement in the inflated configuration. In some embodiments, the first inflatable member 20a and the second inflatable member 20b may generally define a plane 19 in the inflated configuration. In some embodiments, the first aperture 50 and/or the second aperture 52 may be generally oriented at a right angle to (e.g., perpendicular to) the plane 19 defined by the first inflatable member 20a and the second inflatable member 20b.

The inflatable orienting element 20 may have a thickness 21 and a width 23 in the inflated configuration. The width 23 may be measured parallel to the plane 19 and the thickness 21 may be measured perpendicular to the plane 19. The width 23 is greater than the thickness 21 in the inflated configuration. In some embodiments, an aspect ratio of the width 23 to the thickness 21 may be at least 2.0 in the inflated configuration. In some embodiments, the aspect ratio of the width 23 to the thickness 21 (e.g., the width 23 divided by the thickness 21) may be at least 2.5 in the inflated configuration. In some embodiments, the aspect ratio of the width 23 to the thickness 21 may be at least 3.0 in the inflated configuration. In some embodiments, the aspect ratio of the width 23 to the thickness 21 may be at least 3.5 in the inflated configuration. In some embodiments, the aspect ratio of the width 23 to the thickness 21 may be at least 4.0 in the inflated configuration. Other configurations are also contemplated.

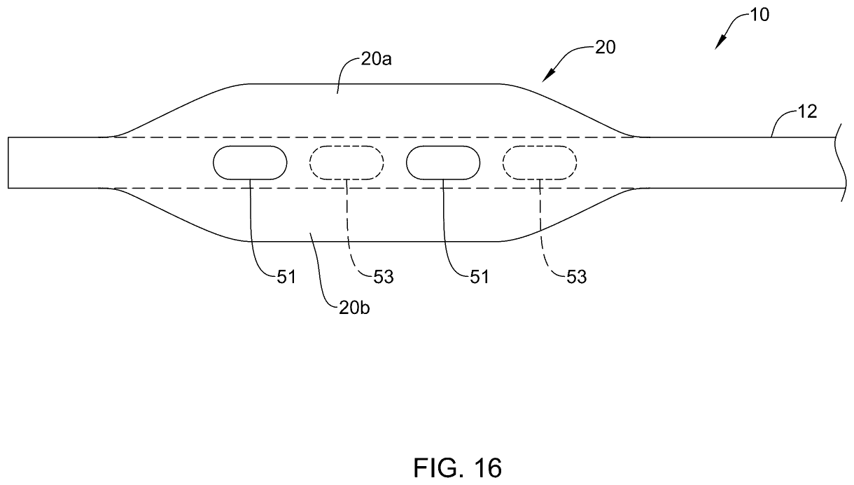

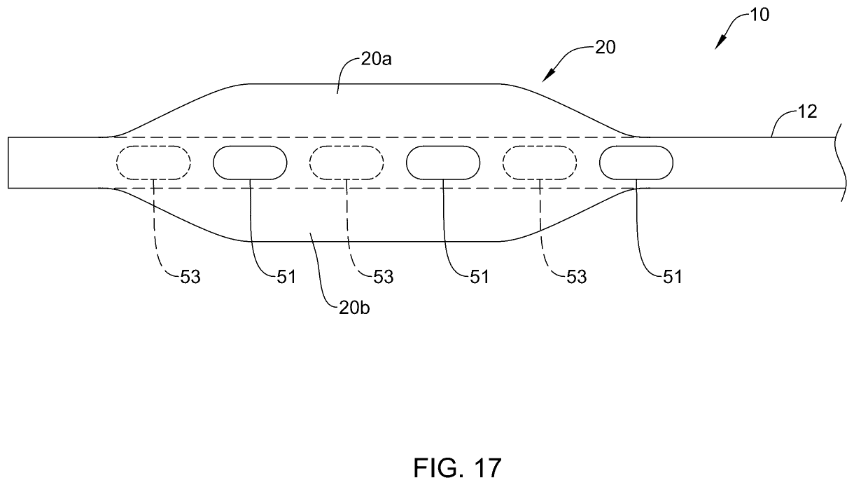

In some embodiments, the self-orienting device 10 and/or the elongate shaft 12 may comprise a plurality of first apertures 51 communicating with the working lumen 24 and opening in a third direction different from the first direction and the second direction, as seen in FIGS. 16-17. In some embodiments, the self-orienting device 10 and/or the elongate shaft 12 may comprise a plurality of second apertures 53 communicating with the working lumen 24 and opening in a fourth direction different from the first direction and the second direction. In some embodiments, the fourth direction may be opposite the third direction. In some embodiments, the plurality of second apertures 53 may be longitudinally spaced apart from and/or longitudinally offset from the plurality of first apertures 51. In some embodiments, the plurality of first apertures 51 may alternate with the plurality of second apertures 53 along the central longitudinal axis and/or along the elongate shaft 12 (e.g., apertures alternating longitudinally—a first aperture, a second aperture, a first aperture, a second aperture, etc.).

In some embodiments, the plurality of first apertures 51 may be disposed between the first inflatable member 20a and the second inflatable member 20b. In some embodiments, a portion of, one of, some of, and/or all of the plurality of first apertures 51 may be disposed proximal and/or distal of the first inflatable member 20a and the second inflatable member 20b. In some embodiments, the plurality of second apertures 53 may be disposed between the first inflatable member 20a and the second inflatable member 20b. In some embodiments, a portion of, one of, some of, and/or all of the plurality of second apertures 53 may be disposed proximal and/or distal of the first inflatable member 20a and the second inflatable member 20b. Other configurations are also contemplated.

In some embodiments, the plurality of first apertures 51 may comprise two first apertures, as seen in FIG. 16. In some embodiments, the plurality of second apertures 53 may comprise two second apertures, as seen in FIG. 16. In some embodiments, the plurality of first apertures 51 may comprise three first apertures, as seen in FIG. 17. In some embodiments, the plurality of second apertures 53 may comprise three second apertures, as seen in FIG. 17. Other configurations, including combinations thereof (e.g., the plurality of first apertures 51 may comprise three first apertures and the plurality of second apertures 53 may comprise two second apertures, the plurality of first apertures 51 may comprise two first apertures and the plurality of second apertures 53 may comprise three second apertures, etc.), are also contemplated.

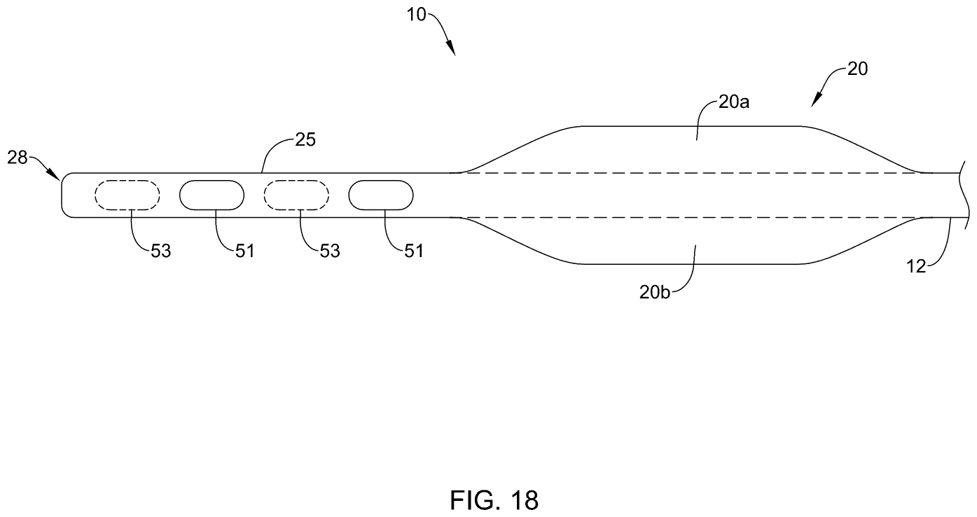

FIG. 18 illustrates selected aspects of an alternative configuration of the self-orienting device 10. In some embodiments, the self-orienting device 10 may comprise structures, configurations, and/or elements as described above. In some embodiments, the self-orienting device 10 and/or the elongate shaft 12 may comprise a distal tail 25 extending distally from the inflatable orienting element 20 to the distal end and/or the distal facing port 28 of the elongate shaft 12. In some embodiments, the distal tail 25 may be at least 15 millimeters long from the inflatable orienting element 20 to the distal end and/or the distal facing port 28 of the elongate shaft 12. In some embodiments, the distal tail 25 may be at least 20 millimeters long from the inflatable orienting element 20 to the distal end and/or the distal facing port 28 of the elongate shaft 12. In some embodiments, the distal tail 25 may be at least 25 millimeters long from the inflatable orienting element 20 to the distal end and/or the distal facing port 28 of the elongate shaft 12. In some embodiments, the distal tail 25 may be at least 30 millimeters long from the inflatable orienting element 20 to the distal end and/or the distal facing port 28 of the elongate shaft 12. In some embodiments, the distal tail 25 may be at least 35 millimeters long from the inflatable orienting element 20 to the distal end and/or the distal facing port 28 of the elongate shaft 12. In some embodiments, the distal tail 25 may be at least 40 millimeters long from the inflatable orienting element 20 to the distal end and/or the distal facing port 28 of the elongate shaft 12.

In some embodiments, the distal tail 25 may be about as long as the inflatable orienting element 20. In some embodiments, the distal tail 25 may be longer than the inflatable orienting element 20. In some embodiments, a length of the distal tail 25 may be at least 50% greater than a length of the inflatable orienting element 20. In some embodiments, the length of the distal tail 25 may be at least 75% greater than the length of the inflatable orienting element 20. In some embodiments, the length of the distal tail 25 may be at least 100% greater than the length of the inflatable orienting element 20. In some embodiments, the length of the distal tail 25 may be at least 150% greater than the length of the inflatable orienting element 20. In some embodiments, the length of the distal tail 25 may be at least 200% greater than the length of the inflatable orienting element 20. In some embodiments, the length of the distal tail 25 may be at least 250% greater than the length of the inflatable orienting element 20. In some embodiments, the length of the distal tail 25 may be at least 300% greater than the length of the inflatable orienting element 20. In some embodiments, the length of the distal tail 25 may be at least 350% greater than the length of the inflatable orienting element 20. In some embodiments, the length of the distal tail 25 may be at least 400% greater than the length of the inflatable orienting element 20. Other configurations are also contemplated.

In some embodiments, the self-orienting device 10 and/or the elongate shaft 12 may comprise a plurality of first apertures 51 communicating with the working lumen 24 and opening in a third direction different from the first direction and the second direction. In some embodiments, the plurality of first apertures 51 may be disposed in the distal tail 25 distal of the inflatable orienting element 20 and proximal of the distal end and/or the distal facing port 28 of the elongate shaft 12.

In some embodiments, the self-orienting device 10 and/or the elongate shaft 12 may comprise a plurality of second apertures 53 communicating with the working lumen 24 and opening in a fourth direction opposite the third direction. In some embodiments, the plurality of second apertures 53 may be disposed in the distal tail 25 distal of the inflatable orienting element 20 and proximal of the distal end and/or the distal facing port 28 of the elongate shaft 12.

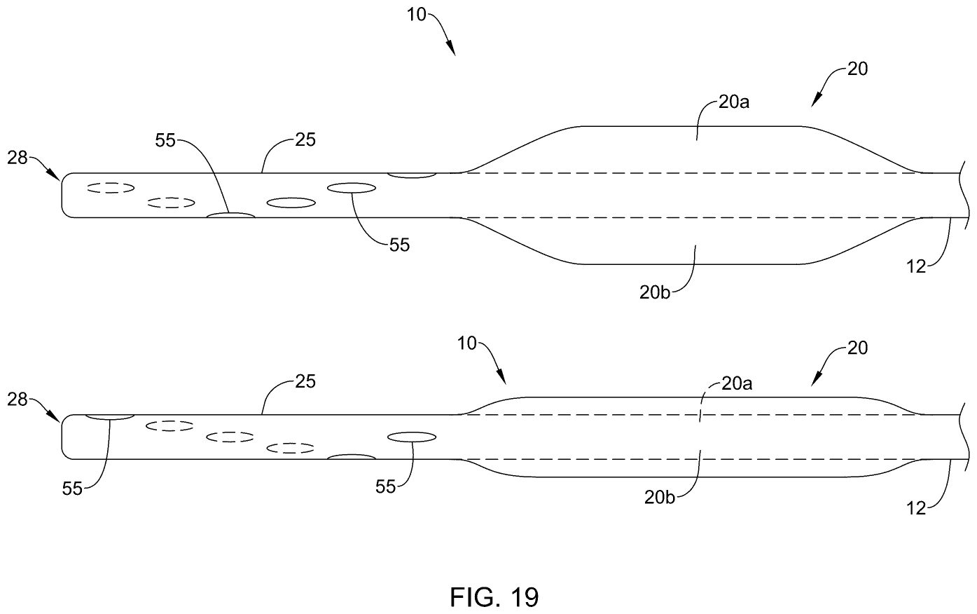

In some embodiments, the self-orienting device 10 and/or the elongate shaft 12 may comprise a plurality of apertures 55 communicating with the working lumen 24 and opening in a plurality of directions different from the first direction and the second direction, as seen in the top and side views shown in FIG. 19. In some embodiments, the plurality of directions may be oriented at a plurality of angles around a circumference of the elongate shaft 12. In some embodiments, the plurality of apertures 55 may comprise six apertures oriented about 60 degrees from each other, as seen in FIG. 19. Other configurations, quantities of apertures, and/or quantities of directions are also contemplated. In some embodiments, the plurality of apertures 55 may comprise 4 apertures, 5 apertures, 6 apertures, 7 apertures, 8 apertures, 9 apertures, 10 apertures, etc. In some embodiments, the plurality of apertures 55 may be oriented about 90 degrees from each other, about 72 degrees from each other, about 60 degrees from each other, about 51 degrees from each other, about 45 degrees from each other, about 40 degrees from each other, about 36 degrees from each other, etc. In some embodiments, none of the plurality of apertures 55 open in the same direction(s) as the first inflatable member 20a and/or the second inflatable member 20b (e.g., none of the plurality of apertures 55 open in the first direction or the second direction). In some embodiments, the plurality of apertures 55 may be disposed in the distal tail 25 distal of the inflatable orienting element 20 and proximal of the distal end and/or the distal facing port 28 of the elongate shaft 12.

In some embodiments, though not expressly illustrated, the self-orienting device 10 and/or the elongate shaft 12 may comprise the plurality of first apertures 51 communicating with the working lumen 24, the plurality of second apertures 53 communicating with the working lumen 24, and/or the plurality of apertures 55 communicating with the working lumen 24 disposed proximal of the inflatable orienting element 20.

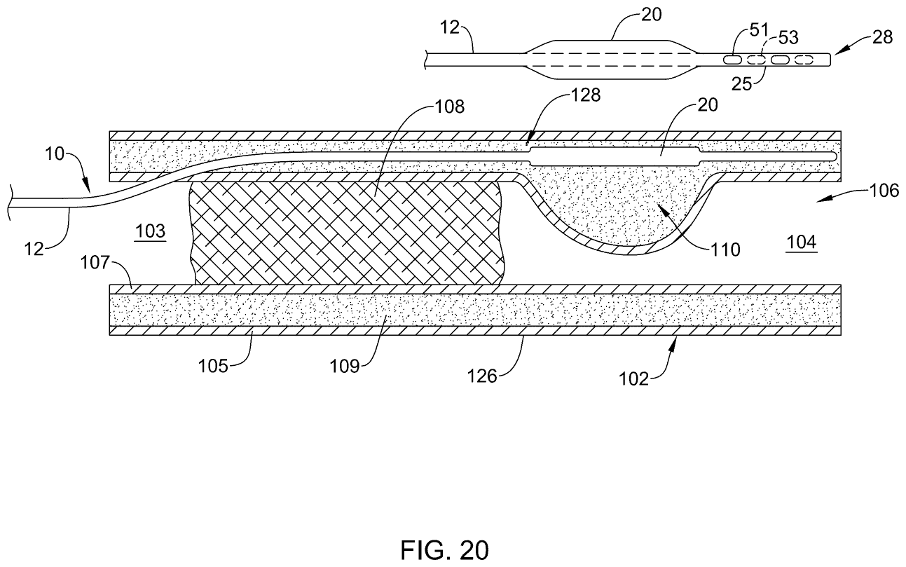

In some cases where the hematoma 110 is present in the subintimal space 128, the distal tail 25 may permit the inflatable orienting element 20 to provide stability and/or orientation relative to the true lumen 106 of the blood vessel 102 while positioning the plurality of first apertures 51 and/or the plurality of second apertures 53 within the distal tail 25 distal of the hematoma 110 and/or any compressed portion of the distal segment 104 of the true lumen 106 of the blood vessel 102, where there is less dissection of the layers of the wall 126 of the blood vessel 102. FIG. 20 illustrates a side view of the self-orienting device 10 disposed within the subintimal space 128 and the distal tail 25 extending distal of the hematoma 110, as well as a top view of the inflatable orienting element 20 and the distal tail 25 to demonstrate the relative position of the plurality of first apertures 51 and/or the plurality of second apertures 53 with respect to the hematoma 110, etc. The positioning shown in FIG. 20 may permit better re-entry into the true lumen 106 of the blood vessel 102 and/or may reduce the need to aspirate the hematoma 110.

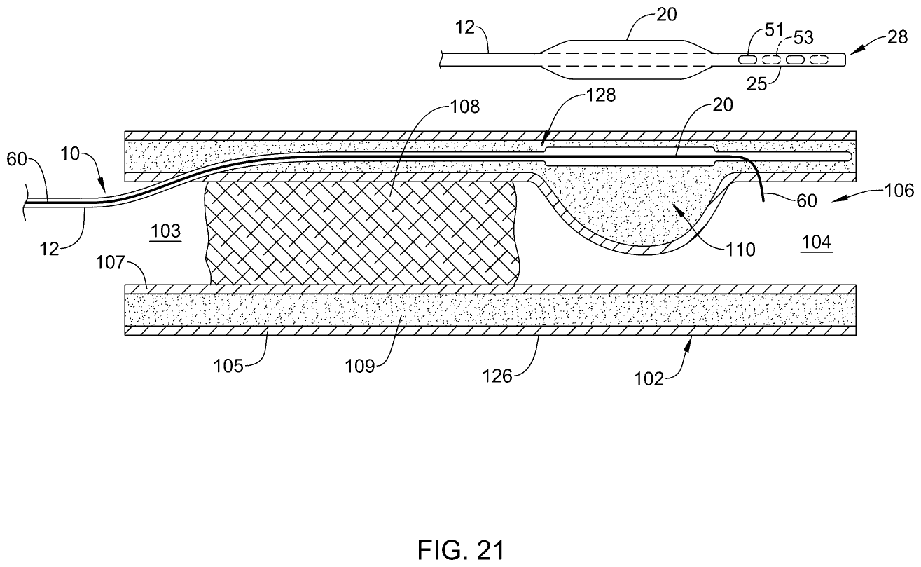

In some embodiments, the plurality of first apertures 51 and/or the plurality of second apertures 53 within the distal tail 25 may be configured to direct the re-entry device 60 into the true lumen 106 of the blood vessel 102 distal of the hematoma 110, as shown in FIG. 21. In some embodiments, a method of treating the total occlusion 108 in the blood vessel 102 may comprise advancing the re-entry device 60 within the working lumen 24 and out one aperture of the plurality of first apertures 51 or the plurality of second apertures 53 into the true lumen 106 of the blood vessel 102 distal of the total occlusion 108 and/or distal of the hematoma 110. In some embodiments, the re-entry device 60 may be disposed within the working lumen 24 prior to advancing the self-orienting device 10 into the subintimal space 128 within the wall 126 of the blood vessel 102. In some embodiments, the re-entry device 60 may be advanced within and/or through the self-orienting device 10 after the self-orienting device 10 has been advanced into the subintimal space 128. Other configurations are also contemplated.

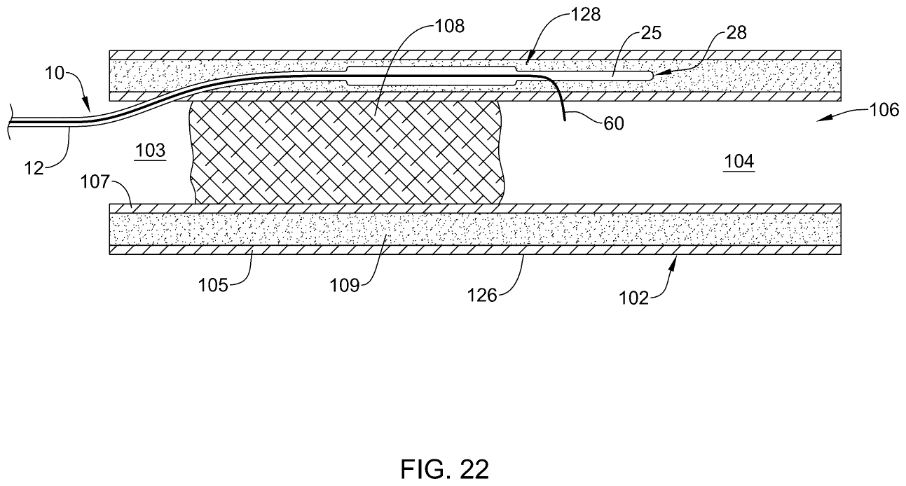

In some embodiments, the self-orienting device 10 comprising the distal tail 25 may be used differently and/or may permit a different method of treating the total occlusion 108 in the blood vessel 102 to be used. In some embodiments, the method may comprise advancing the self-orienting device 10 into the subintimal space 128 within the wall 126 of the blood vessel 102 adjacent the total occlusion 108. The method may comprise positioning the inflatable orienting element 20 radially outward of the total occlusion 108 within the subintimal space 128 such that the inflatable orienting element 20 longitudinally overlaps the total occlusion 108 (e.g., the inflatable orienting element 20 is disposed alongside the total occlusion 108) and the distal tail 25 extends distal of the total occlusion 108 within the subintimal space 128, as seen in FIG. 22. In some embodiments, the method may comprise aspirating blood and/or the hematoma 110 from within the subintimal space 128 through the working lumen 24. The method may comprise inflating the inflatable orienting element 20 within the subintimal space 128 such that the first inflatable member 20a and the second inflatable member 20b cooperate with the wall 126 (e.g., the layers of the wall 126) of the blood vessel 102 to orient the plurality of first apertures 51 or the plurality of second apertures 53, where present, toward the true lumen 106 of the blood vessel 102.

In some embodiments, the re-entry device 60 may be disposed within the working lumen 24 prior to advancing the self-orienting device 10 into the subintimal space 128 within the wall 126 of the blood vessel 102. In some embodiments, the re-entry device 60 may be advanced within and/or through the self-orienting device 10 after the self-orienting device 10 has been advanced into the subintimal space 128. Other configurations are also contemplated. In some embodiments, aspirating blood and/or the hematoma 110 from within the subintimal space 128 occurs while the re-entry device 60 is disposed within the working lumen 24. In some embodiments, aspirating blood and/or the hematoma 110 from within the subintimal space 128 includes aspirating blood and/or the hematoma 110 through the distal facing port 28 and/or the plurality of first apertures 51 and/or the plurality of second apertures 53. In some embodiments, aspirating blood and/or the hematoma 110 from within the subintimal space 128 occurs while positioning the inflatable orienting element 20 radially outward of the total occlusion 108 within the subintimal space 128. In some embodiments, aspirating blood from within the subintimal space 128 while positioning the inflatable orienting element 20 radially outward of the total occlusion 108 may prevent the hematoma 110 from forming.

In some embodiments, the method may comprise advancing the re-entry device 60 within the working lumen 24 and out one aperture of the plurality of first apertures 51 or the plurality of second apertures 53 into the true lumen 106 of the blood vessel 102 distal of the total occlusion 108, as seen in FIGS. 21-22.

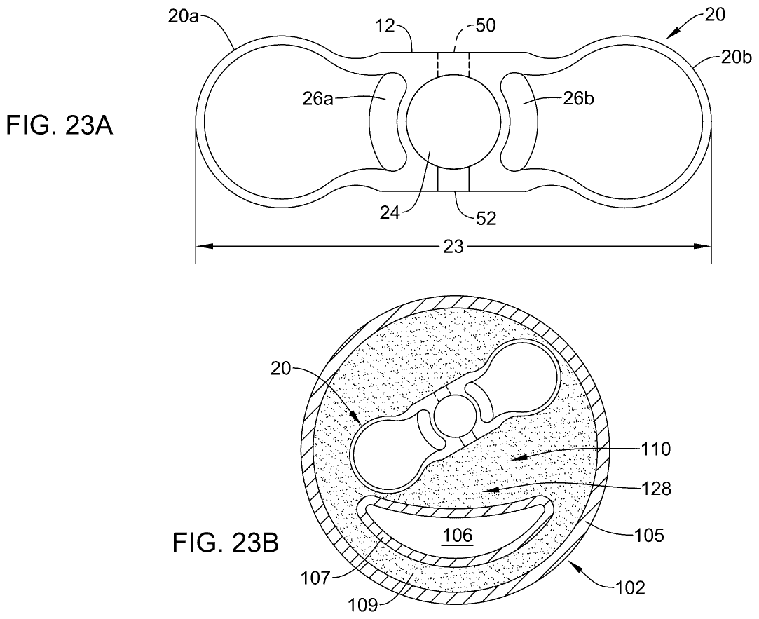

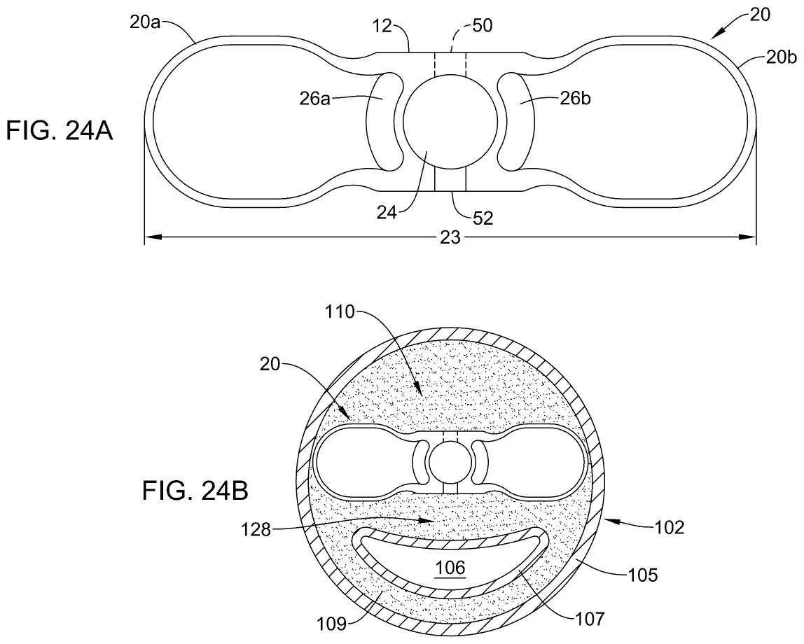

In some cases, the hematoma 110 may become so large that the inflatable orienting element 20, shown in FIG. 23A, may be too small and become misoriented within the subintimal space 128 and/or the hematoma 110, as seen in FIG. 23B. For example, the width 23 of the inflatable orienting element 20 may be about 2.9 millimeters (about 0.114 inches). If a large hematoma forms, the width 23 of the inflatable orienting element 20 may be inadequate to properly orient the plurality of first apertures 51 or the plurality of second apertures 53 toward the true lumen 106 of the blood vessel 102, which may cause failure of the re-entry procedure and/or may cause injury to the blood vessel 102. To address the formation of large hematoma, the width 23 of the inflatable orienting element 20 may be increased, as shown in FIG. 24A. In some embodiments, the width 23 of the inflatable orienting element 20 may be increased to about 3.5 millimeters (about 0.138 inches). Such an increase in the width 23 may also increase the aspect ratio of the inflatable orienting element 20. When used in a large hematoma, the inflatable orienting element 20 having the width 23 of about 3.5 millimeters (about 0.138 inches) may better fit the subintimal space 128 and provide better orientation of the plurality of first apertures 51 or the plurality of second apertures 53 toward the true lumen 106 of the blood vessel 102, as shown in FIG. 24B. In some embodiments, the width 23 of the inflatable orienting element 20 may be increased to about 4.2 millimeters (about 0.165 inches). Other configurations and/or dimensions for the width 23 are also contemplated. For example, the width 23 may be selected based on the size of the blood vessel 102 being treated. In another example, the width 23 may be selected after the large hematoma has formed based on the size of the large hematoma itself. These are only examples and are not intended to be limiting.

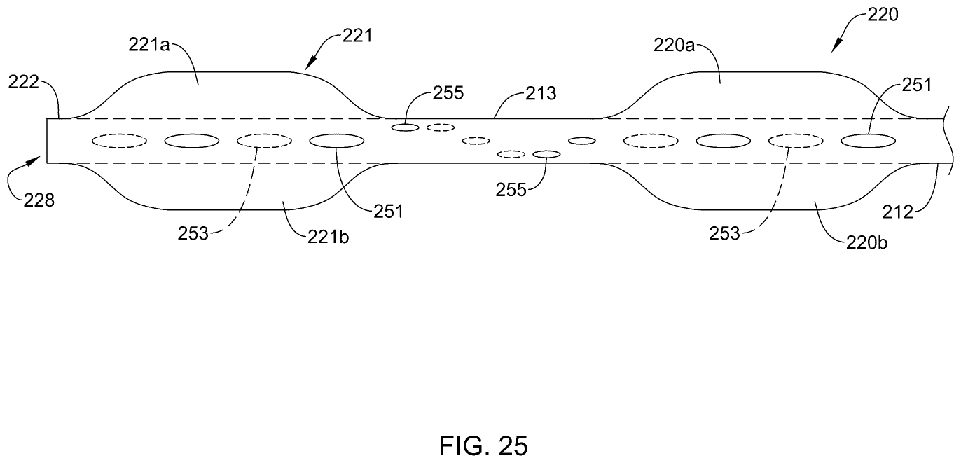

In some embodiments, the apparatus for treating the total occlusion 108 may comprise a self-orienting device 210, as seen in FIG. 25. The self-orienting device 210 may comprise an elongate shaft 212 extending from the manifold 14 (e.g., FIG. 5) to a first inflatable orienting element 220 disposed on and/or mounted on a distal portion of the elongate shaft 212. In some embodiments, the distal portion of the elongate shaft 212 may extend through the first inflatable orienting element 220 and/or distal of the first inflatable orienting element 220 to form an intermediate segment 213 extending from the first inflatable orienting element 220 to a second inflatable orienting element 221. The second inflatable orienting element 221 may be axially and/or longitudinally spaced apart from the first inflatable orienting element 220. The second inflatable orienting element 221 may be disposed distal of the first inflatable orienting element 220. In some embodiments, the elongate shaft 212 and/or the intermediate segment 213 may extend through the second inflatable orienting element 221 to form a distal tip 222 extending distal of the second inflatable orienting element 221.

As in the apparatus above, the self-orienting device 210 and/or the elongate shaft 212 may comprise a working lumen 24 (e.g., FIG. 5) extending from the manifold 14 to a distal facing port 228 at a distal end of the elongate shaft 212 and/or the distal tip 222. In some embodiments, the manifold 14 may comprise a first port 30 (e.g., FIG. 5) in communication with the working lumen 24. In some embodiments, the first port 30 may be oriented generally parallel to the working lumen 24. In some embodiments, the first port 30 may be coaxially aligned with the working lumen 24. Other configurations are also contemplated. The first port 30 and/or the working lumen 24 may be sized and configured to slidably receive a medical device (e.g., a guidewire, a re-entry device, etc.). In some embodiments, the first port 30 may be considered a device port. In some embodiments, the medical device (e.g., the guidewire, the re-entry device, etc.) may be rotatable within the first port 30 and/or the working lumen 24. In some alternative configurations, the medical device (e.g., the guidewire, the re-entry device, etc.) may be nonrotatable within the first port 30 and/or the working lumen 24.

In some embodiments, the manifold 14 may comprise a second port 34 (e.g., FIG. 5) in fluid communication with the working lumen 24. In some embodiments, the second port 34 may be oriented at an oblique angle to the working lumen 24. Other configurations are also contemplated. In some embodiments, the second port 34 may be useful for transferring fluid to and/or from the working lumen 24 with and/or without the medical device (e.g., the guidewire, the re-entry device, etc.) disposed within the first port 30 and/or the working lumen 24. In some embodiments, the second port 34 may be useful for aspiration of the hematoma 110, as described herein. In some embodiments, the second port 34 may be considered an aspiration port. The second port 34 may be configured to be fluidly connected to a source of suction.

In some embodiments, the self-orienting device 210 and/or the elongate shaft 212 may be configured to be advanced over a guidewire for delivery to a remote location in the vasculature of a patient (e.g., to and/or adjacent the total occlusion 108, for example). In some embodiments, the self-orienting device 210 and/or the elongate shaft 212 may be configured as an over-the-wire (OTW) catheter having the working lumen 24 extending through the entire length of the self-orienting device 210 and/or the elongate shaft 212 from the distal facing port 228 at the distal tip 222 to the first port 30 in the manifold 14. In some embodiments, the distal facing port 228 may be disposed at a distalmost extent of the self-orienting device 210 and/or the elongate shaft 212.

The self-orienting device 210 and/or the elongate shaft 212 may comprise an inflation lumen 26 (e.g., FIG. 5) in fluid communication with the inflatable orienting element 20 and the manifold 14. In some embodiments, the manifold 14 may comprise a third port 36 (e.g., FIG. 5) in fluid communication with the inflation lumen 26. The third port 36 may be configured to fluidly connect to a source of inflation fluid (not shown). In some embodiments, the third port 36 may be considered an inflation port.

In some embodiments, the first inflatable orienting element 220 may comprise a first inflatable member 220a extending laterally from a central longitudinal axis of the elongate shaft 212 in a first direction, and a second inflatable member 220b extending laterally from the central longitudinal axis of the elongate shaft 212 in a second direction different from the first direction. In some embodiments, the second direction may be opposite the first direction. In some embodiments, the first inflatable member 220a and/or the second inflatable member 220b may be integrally formed and/or monolithically formed with the elongate shaft 212. In some embodiments, an outer surface of the elongate shaft 212 may be exposed to an exterior of the self-orienting device 210 between the first inflatable member 220a and the second inflatable member 220b. Other configurations are also contemplated.

In some embodiments, the self-orienting device 210 and/or the elongate shaft 212 may comprise a first plurality of apertures 251 communicating with the working lumen 24 and opening to the exterior of the self-orienting device 210 and/or the elongate shaft 212 in a third direction different from the first direction and the second direction. In some embodiments, the third direction may be substantially perpendicular to the first direction and the second direction. Other configurations are also contemplated. In some embodiments, the first plurality of apertures 251 may be disposed between a proximal end of the first inflatable orienting element 220 and a distal end of the first inflatable orienting element 220.

In some embodiments, the self-orienting device 10 and/or the elongate shaft 12 may comprise a first aperture (e.g., the first aperture 50 of FIGS. 5 and 13-15) communicating with the working lumen 24 and opening to the exterior of the self-orienting device 210 and/or the elongate shaft 212 in a third direction different from the first direction and the second direction. In some embodiments, the third direction may be substantially perpendicular to the first direction and the second direction. Other configurations are also contemplated. In some embodiments, the first aperture may be disposed between a proximal end of the first inflatable orienting element 220 and a distal end of the first inflatable orienting element 220.

In some embodiments, the self-orienting device 210 and/or the elongate shaft 212 may comprise a second plurality of apertures 253 communicating with the working lumen 24 and opening to the exterior of the self-orienting device 210 and/or the elongate shaft 212 in a fourth direction different from the first direction and the second direction. In some embodiments, the fourth direction may be different from the third direction. In some embodiments, the fourth direction may be opposite the third direction. In some embodiments, the fourth direction may be substantially perpendicular to the first direction and the second direction. Other configurations are also contemplated. In some embodiments, the second plurality of apertures 253 may be disposed between the proximal end of the first inflatable orienting element 220 and the distal end of the first inflatable orienting element 220. In some embodiments, the second plurality of apertures 253 may be disposed between the proximal end of the first inflatable orienting element 220 and the distal end of the first inflatable orienting element 220 at a location longitudinally offset from and/or longitudinally spaced apart from the first plurality of apertures 251. In some embodiments, the second plurality of apertures 253 may be disposed on an opposite side of the elongate shaft 212 from the first plurality of apertures 253.

In some embodiments, the self-orienting device 210 and/or the elongate shaft 212 may comprise a second aperture (e.g., the second aperture 52 of FIGS. 5 and 13-15) communicating with the working lumen 24 and opening to the exterior of the self-orienting device 210 and/or the elongate shaft 212 in a fourth direction different from the first direction and the second direction. In some embodiments, the fourth direction may be opposite the third direction. In some embodiments, the fourth direction may be substantially perpendicular to the first direction and the second direction. Other configurations are also contemplated. In some embodiments, the second aperture may be disposed between the proximal end of the first inflatable orienting element 220 and the distal end of the first inflatable orienting element 220. In some embodiments, the second aperture may be disposed between the proximal end of the first inflatable orienting element 220 and the distal end of the first inflatable orienting element 220 at a location longitudinally offset from and/or longitudinally spaced apart from the first aperture. In at least some embodiments, the second aperture may be disposed on an opposite side of the elongate shaft 212 from the first aperture.

In some embodiments, the elongate shaft 212 may also include a first radiopaque marker (not shown) located proximate the first plurality of apertures 251 and/or the first aperture to provide an indication of the location of the first plurality of apertures 251 and/or the first aperture under fluoroscopy. In some embodiments, the elongate shaft 212 may include a second radiopaque marker (not shown) located proximate the second plurality of apertures 253 and/or the second aperture to provide an indication of the location of the second plurality of apertures 253 and/or the second aperture under fluoroscopy. Other configurations are also contemplated.

In some embodiments, the second inflatable orienting element 221 may comprise a first inflatable member 221a extending laterally from a central longitudinal axis of the elongate shaft 212 in a first direction, and a second inflatable member 221b extending laterally from the central longitudinal axis of the elongate shaft 212 in a second direction different from the first direction. In some embodiments, the second direction may be opposite the first direction. In some embodiments, the first inflatable member 221a and/or the second inflatable member 221b may be integrally formed and/or monolithically formed with the elongate shaft 212. In some embodiments, an outer surface of the elongate shaft 212 may be exposed to an exterior of the self-orienting device 210 between the first inflatable member 221a and the second inflatable member 221b. Other configurations are also contemplated.

In some embodiments, the first plurality of apertures 251 may be disposed between a proximal end of the second inflatable orienting element 221 and a distal end of the second inflatable orienting element 221 in addition to or in place of the first plurality of apertures 251 disposed between the proximal end of the first inflatable orienting element 220 and the distal end of the first inflatable orienting element 220.

In some embodiments, the self-orienting device 210 and/or the elongate shaft 212 may comprise a first set of the first plurality of apertures 251 disposed between the proximal end of the first inflatable orienting element 220 and the distal end of the first inflatable orienting element 220 and a second set of the first plurality of apertures 251 disposed between the proximal end of the second inflatable orienting element 221 and the distal end of the second inflatable orienting element 221. In some embodiments, the first set of the first plurality of apertures 251 may be axially and/or longitudinally spaced apart from the second set of the first plurality of apertures 251 by the intermediate segment 213.

In some embodiments, the first aperture may be disposed between a proximal end of the second inflatable orienting element 221 and a distal end of the second inflatable orienting element 221 in addition to or in place of the first aperture disposed between the proximal end of the first inflatable orienting element 220 and the distal end of the first inflatable orienting element 220.

In some embodiments, the second plurality of apertures 253 may be disposed between the proximal end of the second inflatable orienting element 221 and the distal end of the second inflatable orienting element 221 in addition to or in place of the second plurality of apertures 253 disposed between the proximal end of the second inflatable orienting element 221 and the distal end of the second inflatable orienting element 221. In some embodiments, the second plurality of apertures 253 may be disposed between the proximal end of the second inflatable orienting element 221 and the distal end of the second inflatable orienting element 221 at a location longitudinally offset from and/or longitudinally spaced apart from the first plurality of apertures 251. In some embodiments, the second plurality of apertures 253 may be disposed on an opposite side of the elongate shaft 112 from the first plurality of apertures 253.

In some embodiments, the self-orienting device 210 and/or the elongate shaft 212 may comprise a first set of the second plurality of apertures 253 disposed between the proximal end of the first inflatable orienting element 220 and the distal end of the first inflatable orienting element 220 and a second set of the second plurality of apertures 253 disposed between the proximal end of the second inflatable orienting element 221 and the distal end of the second inflatable orienting element 221. In some embodiments, the first set of the second plurality of apertures 253 may be axially and/or longitudinally spaced apart from the second set of the second plurality of apertures 253 by the intermediate segment 213.

In some embodiments, the second aperture may be disposed between the proximal end of the second inflatable orienting element 221 and the distal end of the second inflatable orienting element 221 in addition to or in place of the second aperture disposed between the proximal end of the second inflatable orienting element 221 and the distal end of the second inflatable orienting element 221. In some embodiments, the second aperture may be disposed between the proximal end of the second inflatable orienting element 221 and the distal end of the second inflatable orienting element 221 at a location longitudinally offset from and/or longitudinally spaced apart from the first aperture. In at least some embodiments, the second aperture may be disposed on an opposite side of the elongate shaft 212 from the first aperture.

In some embodiments, in addition or alternatively to the various configurations of apertures described herein, the self-orienting device 210 and/or the elongate shaft 212 may comprise a plurality of apertures 255 communicating with the working lumen 24 and opening in a plurality of directions different from the first direction and/or the second direction. The plurality of apertures 255 may be disposed in and/or along the intermediate segment 213. In some embodiments, the plurality of apertures 255 may be disposed axially and/or longitudinally between the first inflatable orienting element 220 and the second inflatable orienting element 221.

In some embodiments, the plurality of directions may be oriented at a plurality of angles around a circumference of the elongate shaft 212 and/or the intermediate segment 213. In some embodiments, the plurality of apertures 255 may comprise six apertures oriented about 60 degrees from each other. Other configurations, quantities of apertures, and/or quantities of directions are also contemplated. In some embodiments, the plurality of apertures 255 may comprise 4 apertures, 5 apertures, 6 apertures, 7 apertures, 8 apertures, 9 apertures, 10 apertures, etc. In some embodiments, the plurality of apertures 255 may be oriented about 90 degrees from each other, about 72 degrees from each other, about 60 degrees from each other, about 51 degrees from each other, about 45 degrees from each other, about 40 degrees from each other, about 36 degrees from each other, etc. In some embodiments, none of the plurality of apertures 255 open in the same direction(s) as the first inflatable member 220a and/or the second inflatable member 220b of the first inflatable orienting element 220 and/or the first inflatable member 221a and/or the second inflatable member 221b of the second inflatable orienting element 221 (e.g., none of the plurality of apertures 255 open in the first direction or the second direction). Other configurations are also contemplated.

The self-orienting device 210 may find several uses. In some embodiments, the self-orienting device 210 may be advanced into the subintimal space 128 (e.g., FIG. 6) and/or may be used in a manner similar to the self-orienting device 10 above. In some embodiments, the first inflatable orienting element 220 may be disposed and/or positioned within the hematoma 110 and the second inflatable orienting element 221 may be disposed proximal of the hematoma 110. In some embodiments, the first inflatable orienting element 220 may be disposed and/or positioned distal of the hematoma 110 and the second inflatable orienting element 221 may be disposed within the hematoma 110. In some embodiments, the intermediate segment 213 may span the hematoma 110 such that the first inflatable orienting element 220 may be disposed and/or positioned distal of the hematoma 110 and the second inflatable orienting element 221 may be disposed proximal of the hematoma 110. The hematoma 110 may be aspirated through the working lumen 24 as described herein. Having at least one of the first inflatable orienting element 220 and/or the second inflatable orienting element 221 disposed outside of the hematoma 110 may provide stability and/or orienting capability for the self-orienting device 210.

In some embodiments, the first plurality of apertures 251, the second plurality of apertures 253 and/or the plurality of apertures 255 may provide a plurality of different orientations and/or pathways to align the re-entry device 60 with the true lumen 106 for advancement toward and/or re-entry into the true lumen 106.

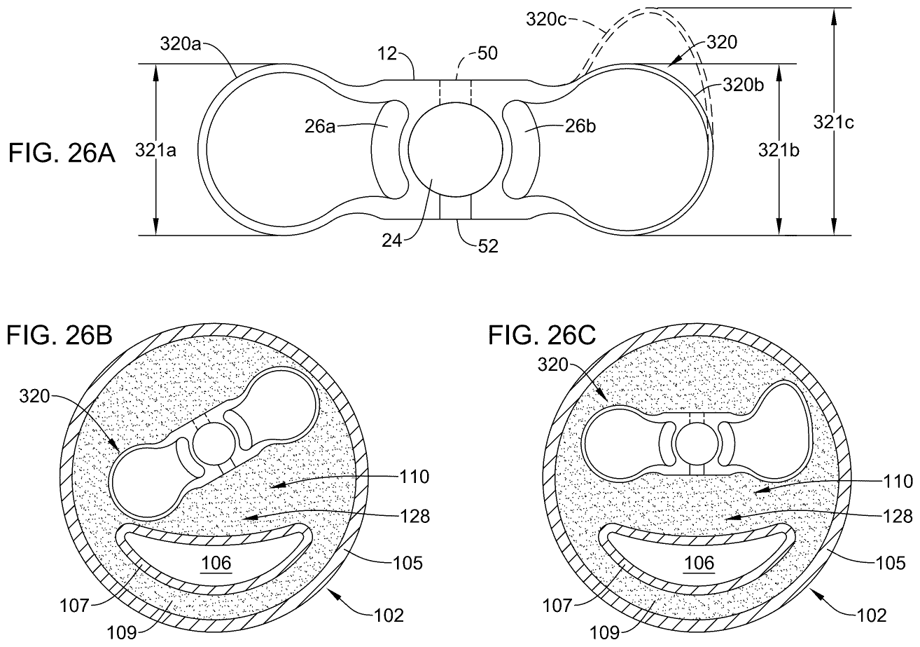

In some embodiments, such as when the subintimal space 128 and/or the dissection path has taken a spiral path around the true lumen 106, the intermediate segment 213 and/or the plurality of apertures 255 may provide a plurality of different orientations and/or pathways to ensure the re-entry device 60 may be aligned with the true lumen 106 as the self-orienting device 210 twists and/or spirals around the true lumen 106. For example, assume that a reference plane extends through the central longitudinal axis of the true lumen 106 and/or the blood vessel 102. After advancing the crossing device 120 and/or the guidewire 121 into the subintimal space 128 as described herein, the dissection path may spiral around the true lumen 106. As such, after the self-orienting device 210 is positioned within the subintimal space 128, the first inflatable orienting element 220 may be oriented (in an end view, or along the central longitudinal axis of the true lumen 106 and/or the blood vessel 102) at a first oblique angle relative to the reference plane extending through the central longitudinal axis of the true lumen 106 and/or the blood vessel 102 and the second inflatable orienting element 221 may be oriented at a second oblique angle different from the first oblique angle relative to the reference plane extending through the central longitudinal axis of the true lumen 106 and/or the blood vessel 102. The intermediate segment 213 may be twisted and/or may spiral around the true lumen 106. However, the presence of the plurality of apertures 255 at a plurality of angles and/or directions around the intermediate segment 213 may ensure that a suitable re-entry angle and/or path is available to advance the re-entry device 60 toward the true lumen. FIG. 26A illustrates selected aspects of an alternative configuration of an inflatable orienting element 320. It will be understood that the inflatable orienting element 320 may be used in place of and/or may be interchanged with the inflatable orienting element 20, the first inflatable orienting element 220 and/or the second inflatable orienting element 221 described herein. The inflatable orienting element 320 may comprise a first inflatable member 320a extending laterally from a central longitudinal axis of the elongate shaft 12 in a first direction, and a second inflatable member 320b extending laterally from the central longitudinal axis of the elongate shaft 12 in a second direction different from the first direction. In some embodiments, the second direction may be opposite the first direction. In some embodiments, the first inflatable member 320a and/or the second inflatable member 320b may be integrally formed and/or monolithically formed with the elongate shaft 12. In some embodiments, an outer surface of the elongate shaft 12 may be exposed to an exterior of the self-orienting device 10 between the first inflatable member 320a and the second inflatable member 320b. Other configurations are also contemplated.

The first inflatable member 320a may have a first thickness 321a. The first inflatable member 320a may have a first wall thickness. The second inflatable member 320b may have a second thickness 321b at a first inflation pressure and a third thickness 321c at a second inflation pressure greater than the first inflation pressure. The second inflatable member 320b may have a second wall thickness less than the first wall thickness of the first inflatable member 320a. The second wall thickness of the second inflatable member 320b may permit the second inflatable member 320b to be expanded to the third thickness 321c if the physician determines that the inflatable orienting element 320 is misaligned with the true lumen 106, as seen in FIG. 26B. When the inflation pressure is increased above the first inflation pressure and/or toward the second inflation pressure, the second wall thickness may expand toward the third thickness 321c, as seen in FIG. 26C. The second inflatable member 320b may push against and/or exert a force against the outermost layer 105, which may cause the inflatable orienting element 320 to rotate within the subintimal space 128 and/or the hematoma 110 toward a position and/or orientation that better aligns the first aperture 50 and/or the second aperture 52 (or at least one of any other apertures that may be present—e.g., the first plurality of apertures 51/251, the second plurality of apertures 53/253, the plurality of aperture 55/255, etc.) with the true lumen 106. Other configurations are also contemplated. In some embodiments, changes in inflation pressure may permit the physician to “fine tune” the orientation of the inflatable orienting element 320 and/or the angle and/or direction of the re-entry device 60 extending therefrom.

In some alternative configurations, the second inflatable member 320b may be formed only with the third thickness 321c such that the at the first inflation pressure, the first inflatable member 320a has the first thickness 321a and the second inflatable member 320b has the third thickness 321c, thereby causing the inflatable orienting element 320 to “tilt” and/or rotate about its own central longitudinal axis. Other configurations are also contemplated.

The materials that can be used for the various components of the apparatus and the various elements thereof disclosed herein may include those commonly associated with medical devices. For simplicity purposes, the following discussion refers to the apparatus. However, this is not intended to limit the devices, components, and methods described herein, as the discussion may be applied to other elements, members, components, or devices disclosed herein, such as, but not limited to, the crossing device, the self-orienting device, the inflatable orienting element, the elongate shaft, etc. and/or elements or components thereof.

In some embodiments, the apparatus and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the apparatus and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-NR and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the apparatus and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some embodiments, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An apparatus for treating a total occlusion in a blood vessel, comprising:
a self-orienting device comprising an elongate shaft, an inflatable orienting element disposed on a distal portion of the elongate shaft, and a manifold disposed at a proximal end of the elongate shaft;
wherein the elongate shaft comprises a working lumen extending from the manifold to a distal facing port at a distal end of the elongate shaft;
wherein the elongate shaft comprises an inflation lumen in fluid communication with the inflatable orienting element and the manifold;
wherein the inflatable orienting element comprises a first inflatable member extending laterally from a central longitudinal axis of the elongate shaft in a first direction and a second inflatable member extending laterally from the central longitudinal axis of the elongate shaft in a second direction different from the first direction;
wherein in a proximal portion of the elongate shaft proximal of the inflatable orienting element, the inflation lumen has a first noncircular cross-sectional shape and the working lumen has a second noncircular cross-sectional shape;
wherein an outer diameter of the proximal portion of the elongate shaft is 0.045 inches or less, a cross-sectional area of the first noncircular cross-sectional shape is at least 0.00013 square inches, and a cross-sectional area of the second noncircular cross-sectional shape is at least 0.00056 square inches.

2. The apparatus of claim 1, wherein the elongate shaft comprises a first aperture communicating with the working lumen and opening in a third direction different from the first direction and the second direction;
wherein the first aperture is disposed between a proximal end of the inflatable orienting element and a distal end of the inflatable orienting element.

3. The apparatus of claim 2, wherein the elongate shaft comprises a second aperture communicating with the working lumen and opening in a fourth direction different from the third direction;
wherein the second aperture is disposed between the proximal end of the inflatable orienting element and the distal end of the inflatable orienting element at a location longitudinally spaced apart from the first aperture.

4. The apparatus of claim 1, wherein the proximal portion of the elongate shaft comprises:
a proximal section wherein the inflation lumen has the first noncircular cross-sectional shape and the working lumen has the second noncircular cross-sectional shape; and
a distal section wherein the inflation lumen and the working lumen each have circular cross-sectional shapes;
wherein a length of the proximal section is at least 50% of an overall length of the proximal portion of the elongate shaft.

5. The apparatus of claim 1, wherein the first noncircular cross-sectional shape is crescent shaped.

6. The apparatus of claim 1, wherein the second noncircular cross-sectional shape has a first maximum extent extending parallel to a major dimension of the first noncircular cross-sectional shape and a second maximum extent oriented perpendicular to the first maximum extent;
wherein the second maximum extent is less than the first maximum extent.

7. The apparatus of claim 6, wherein the first maximum extent is greater than the major dimension.

8. The apparatus of claim 1, wherein the inflatable orienting element is a first inflatable orienting element and the self-orienting device further comprises a second inflatable orienting element axially spaced apart from the first inflatable orienting element, the second inflatable orienting element comprising a first inflatable member extending laterally from a central longitudinal axis of the elongate shaft in the first direction and a second inflatable member extending laterally from the central longitudinal axis of the elongate shaft in the second direction.

9. The apparatus of claim 8, further comprising an intermediate segment disposed between the first inflatable orienting element and the second inflatable orienting element, the intermediate segment comprising a plurality of apertures opening in a plurality of directions different from the first direction and the second direction.

10. The apparatus of claim 1, further comprising a re-entry device configured to be slidably advanced within the working lumen;
wherein the elongate shaft is configured to direct the re-entry device toward a true lumen of the blood vessel at a position distal of the total occlusion.

11. An apparatus for treating a total occlusion in a blood vessel, comprising:
a self-orienting device comprising an elongate shaft, an inflatable orienting element disposed on a distal portion of the elongate shaft, and a manifold disposed at a proximal end of the elongate shaft;
wherein the elongate shaft comprises a working lumen extending from the manifold to a distal facing port at a distal end of the elongate shaft;
wherein the elongate shaft comprises an inflation lumen in fluid communication with the inflatable orienting element and the manifold;
wherein the inflatable orienting element comprises a first inflatable member extending laterally from a central longitudinal axis of the elongate shaft in a first direction and a second inflatable member extending laterally from the central longitudinal axis of the elongate shaft in a second direction opposite the first direction;
wherein the elongate shaft comprises a distal tail extending distally from the inflatable orienting element to the distal end of the elongate shaft, wherein the distal tail is at least 15 millimeters long;
wherein the elongate shaft comprises a plurality of first apertures communicating with the working lumen and opening in a third direction different from the first direction and the second direction, wherein the plurality of first apertures is disposed in the distal tail distal of the inflatable orienting element and proximal of the distal end.

12. The apparatus of claim 11, wherein the elongate shaft comprises a plurality of second apertures communicating with the working lumen and opening in a fourth direction opposite the third direction;
wherein the plurality of second apertures is disposed in the distal tail distal of the inflatable orienting element and proximal of the distal end.

13. The apparatus of claim 12, wherein the plurality of second apertures is longitudinally spaced apart from the plurality of first apertures.

14. The apparatus of claim 11, wherein in a proximal portion of the elongate shaft proximal of the inflatable orienting element, the inflation lumen has a first noncircular cross-sectional shape and the working lumen has a second noncircular cross-sectional shape;
wherein an outer diameter of the proximal portion of the elongate shaft is 0.045 inches or less, a cross-sectional area of the first noncircular cross-sectional shape is at least 0.00013 square inches, and a cross-sectional area of the second noncircular cross-sectional shape is at least 0.00056 square inches.

15. A method of treating a total occlusion in a blood vessel, comprising:
advancing a self-orienting device comprising an elongate shaft, an inflatable orienting element disposed on a distal portion of the elongate shaft, and a manifold disposed at a proximal end of the elongate shaft into a subintimal space within a wall of the blood vessel adjacent the total occlusion;
wherein the elongate shaft comprises a working lumen extending from the manifold to a distal facing port at a distal end of the elongate shaft, and an inflation lumen in fluid communication with the inflatable orienting element and the manifold;
wherein the inflatable orienting element comprises a first inflatable member extending laterally from a central longitudinal axis of the elongate shaft in a first direction and a second inflatable member extending laterally from the central longitudinal axis of the elongate shaft in a second direction different from the first direction;
wherein the elongate shaft comprises a distal tail extending distally from the inflatable orienting element to the distal end of the elongate shaft, wherein the distal tail is at least 15 millimeters long;
wherein the elongate shaft comprises a plurality of first apertures communicating with the working lumen and opening in a third direction different from the first direction and the second direction, wherein the plurality of first apertures is disposed in the distal tail distal of the inflatable orienting element and proximal of the distal end;
positioning the inflatable orienting element radially outward of the total occlusion within the subintimal space such that the inflatable orienting element longitudinally overlaps the total occlusion and the distal tail extends distal of the total occlusion within the subintimal space;
aspirating blood from within the subintimal space through the working lumen; and
inflating the inflatable orienting element within the subintimal space such that the first inflatable member and the second inflatable member cooperate with the wall of the blood vessel to orient the plurality of first apertures toward a true lumen of the blood vessel.

16. The method of claim 15, further comprising:
advancing a re-entry device within the working lumen and out one aperture of the plurality of first apertures into the true lumen of the blood vessel.

17. The method of claim 16, wherein the re-entry device is disposed within the working lumen prior to advancing the self-orienting device into a subintimal space within a wall of the blood vessel adjacent the total occlusion.

18. The method of claim 17, wherein aspirating blood from within the subintimal space occurs while the re-entry device is disposed within the working lumen.

19. The method of claim 15, wherein aspirating blood from within the subintimal space includes aspirating blood through the distal facing port and the plurality of first apertures.

20. The method of claim 15, wherein aspirating blood from within the subintimal space occurs while positioning the inflatable orienting element radially outward of the total occlusion within the subintimal space.
